(19) United States
(12) Patent Application Publication
(10) Pub. No.: US 2024/0263201 A1
(43) Pub. Date: Aug. 8, 2024

(54) BIOLOGICAL METHOD FOR OBTAINING MONOMERS COMPRISING AN ETHYLENIC UNSATURATION BY BIOCONVERSION OF A BIO-SOURCED COMPOUND COMPRISING AT LEAST ONE NITRILE FUNCTION

(71) Applicant: SPCM SA, ANDREZIEUX BOUTHEON (FR)

(72) Inventors: Cédrick FAVERO, ANDREZIEUX BOUTHEON (FR); Johann KIEFFER, ANDREZIEUX BOUTHEON (FR)

(21) Appl. No.: 18/565,838

(22) PCT Filed: Jul. 8, 2022
(86) PCT No.: PCT/EP2022/069156

§ 371 Date: Nov. 30, 2023

(30) Foreign Application Priority Data
Jul. 9, 2021 (FR) FR2107485

(51) Int. Cl. C12P 13/02; C04B 24/26; C07C 57/04; C07C 233/09; C08F 220/56; C09K 8/035; C09K 8/467; C09K 8/588; C09K 8/68; C12P 7/40; D21H 17/45

(52) U.S. Cl. CPC C12P 13/02; C04B 24/2652; C07C 57/04; C07C 233/09; C08F 220/56; C09K 8/035; C09K 8/467; C09K 8/588; C09K 8/68; C12P 7/40; D21H 17/455

(57) ABSTRACT

A biological method is for obtaining an MO monomer including an ethylenic unsaturation by bioconversion of a CN compound including at least one nitrile function. The CN compound is at least partially renewable and non-fossil. The biological method includes at least one step of enzymatic bioconversion of the CN compound in the presence of a biocatalyst including at least one enzyme.

### FIELD OF THE INVENTION

The present invention relates to a biological method for obtaining a monomer comprising an ethylenic unsaturation by bioconversion of a bio-sourced compound comprising at least one nitrile function, preferably (meth)acrylonitrile or bio-3-hydroxypropionitrile, said method comprising at least one enzymatic bioconversion step. In particular, the method can be used to obtain bio-sourced (meth)acrylamide, ammonium (meth)acrylate and (meth)acrylic acid. The invention also relates to a bio-sourced polymer obtained from at least one of the monomers bio-sourced and bio-obtained according to the invention. Lastly, the invention relates to the use of the invention's bio-sourced polymers in various technical fields.

### PRIOR ART

Ethylenically unsaturated monomers, such as acrylamide and acrylic acid, or salts thereof, are widely used in manufacturing water-soluble polymers. Acrylamide and acrylic acid, specifically ammonium acrylate (which can then be easily converted to acrylic acid) can be synthesized enzymatically with biocatalysts, such as microorganisms containing enzymes.

Nitrile hydratase enzymes are known to catalyze the hydration of nitriles directly into corresponding amides. Nitrilase enzymes are known to catalyze the hydration of nitriles into corresponding acrylic acid salts. Amidase enzymes are known to catalyze the hydration of amides into corresponding carboxylic acid salts. In all cases, water serves as a solvent and reagent for the enzymatic reaction. The following diagram summarizes these various possibilities (ACN: acrylonitrile, AM: acrylamide, AA salts: acrylic acid salts).

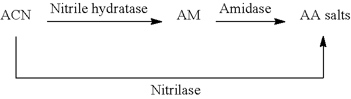

Acrylonitrile is currently produced by an ammoxidation method, commonly known as the SOHIO method, by reaction between propylene (propene) and ammonia, as described in U.S. Pat. No. 2,904,580.

Propylene is a fossil-based olefin, and is currently produced by steam cracking of naphtha, itself derived from crude oil refining. More recently, and with the advent of shale gas production, various propane dehydrogenation methods have been introduced to produce propylene.

Fossil-based propylene contains various impurities, which remain or are transformed by the ammoxidation method.

Impurities in acrylonitrile are known to impact the various methods of bioconversion into (meth)acrylamide or ammonium (meth)acrylate. For example, document JP 11-123098 describes that enzymatic activity is strongly impacted by the presence of hydrocyanic acid in acrylonitrile. Consequently, it is necessary to increase the dose of enzyme to counteract this drop in activity. The presence of acrolein produces monomers that are unsuitable for the polymerization of high molecular weight polymers because they act as cross-linking agents.

The existing literature describes the importance of having a high purity acrylamide in order to obtain high performance polymers, generally of high molecular weight and free of coloration or “fish eyes”, otherwise known as cross-linked or micro-branched polymer particles.

In order to counteract these various drawbacks, several players have sought to purify acrylonitrile. For example, U.S. Pat. Nos. 4,208,329 and 5,969,175 describe methods for purifying acrylonitrile by treatment with ion exchange resins to limit the concentration of oxazole and acrolein.

Other strategies have been adopted with the aim of obtaining a higher purity acrylamide, such as purification of acrylamide. Document EP 3 736 262 A1 describes a method for purifying acrylamide with activated carbon.

Ammonium acrylate is usually obtained by neutralizing acrylic acid with aqueous ammonia. Purification of acrylic acid has been widely described, for example in U.S. Pat. No. 6,541,665.

The problem the invention proposes to resolve is to propose a new and improved method for producing ethylenically unsaturated monomers, such as acrylamide and ammonium acrylate.

### SUMMARY OF THE INVENTION

Quite surprisingly, the Applicant has observed that using a CN compound comprising at least one nitrile function that is at least partially renewable and fossil-based in a method for obtaining an MO monomer comprising an ethylenic unsaturation, said method being a biological method comprising at least one enzymatic bioconversion step, can be used to substantially reduce the consumption of biocatalyst and to increase the recycling rate of said biocatalyst.

In the whole invention, the CN compound comprising at least one nitrile function is preferably (meth)acrylonitrile or 3-hydroxypropionitrile. Preferably, it comprises a single nitrile function. Without seeking to be bound by any particular theory, the Applicant raises the possibility that the different nature of the impurities between a compound comprising at least one fossil-based nitrile function and a compound comprising at least one renewable and non-fossil nitrile function is the cause of these unexpected technical effects.

The first object of the invention is a biological method for obtaining an MO monomer comprising an ethylenic unsaturation by bioconversion of a CN compound comprising at least one nitrile function, said CN compound being at least partially renewable and non-fossil, said biological method comprising at least one enzymatic bioconversion step in the presence of a biocatalyst comprising at least one enzyme.

In the whole invention, the MO monomer comprising an ethylenic unsaturation is preferably (meth)acrylamide, ammonium (meth)acrylate or (meth)acrylic acid.

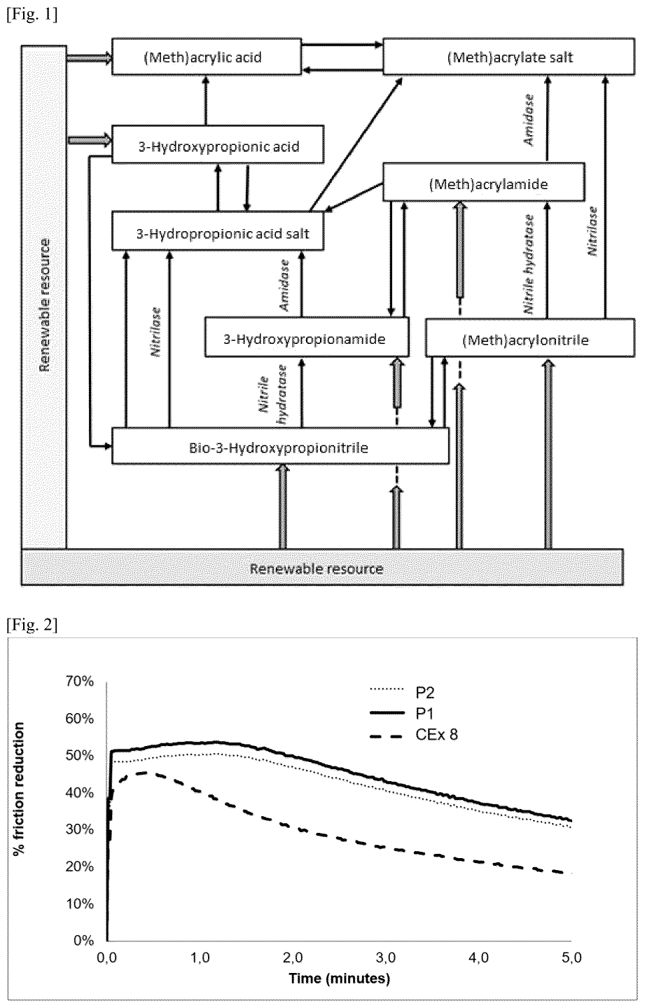

In the whole invention, reference may be made to FIG. 1, which details, inter alia, in a general diagram, the various ways of obtaining the monomers according to the invention.

Another object of the invention is a bio-(meth)acrylamide obtained by bioconversion of a CN compound comprising at least one nitrile function according to a biological method comprising at least one enzymatic bioconversion step, said CN compound comprising at least one nitrile function that is at least partially renewable and non-fossil. “Bio-(meth)acrylamide” means bio-sourced-(meth)acrylamide.

Another object of the invention is a bio-(meth)acrylate salt obtained by bioconversion of a CN compound comprising at least one nitrile function according to a biological method comprising at least one enzymatic bioconversion step, said CN compound comprising at least one nitrile function that is at least partially renewable and non-fossil. “Bio-(meth)acrylate” means bio-sourced-(meth)acrylate.

Another object of the invention is a polymer obtained by polymerization of at least one monomer obtained by the method according to the invention, or obtained by polymerization of at least one bio-(meth)acrylamide or at least one bio-(meth)acrylate salt or a bio-(meth)acrylic acid according to the invention. A further object of the invention is using the polymer according to the invention in various technical fields. “Bio-(meth)acrylic acid” means bio-sourced-(meth)acrylic acid.

With the present invention, it is possible to achieve environmental objectives inherent in new technical innovations. In the present case, combining a bio-method with use of renewable and non-fossil raw material allows to substantially optimize the bioconversion process. It also allows to obtain polymerizable bio-sourced-monomers which deliver unexpectedly improved performances.

Another advantage of the process of the invention is that it allows to reduce the residual amount of CN compound (compound comprising at least one nitrile function).

Moreover, the polymers obtained by polymerization of the MO monomers are more easily biodegradable than polymers with monomers having a fossil origin. They also exhibit improved performance in terms of drainage and retention, improved solubility as compared to fossil monomers. They also are more resistant to chemical degradation, and the friction reduction of injection fluids prepared with such polymers is improved.

### DETAILED DESCRIPTION OF THE INVENTION

In the context of the invention, the terms “renewable and non-fossil” are used to designate the origin of a chemical compound derived from biomass or from synthesis gas (syngas), i.e. resulting from one or more chemical transformations carried out on one or more natural and non-fossil raw materials. The terms “bio-sourced” or “bio-resourced” can also be used to characterize the renewable and non-fossil origin of a chemical compound. The renewable and non-fossil origin of a compound includes renewable and non-fossil raw materials stemming from the circular economy, which have been previously recycled, once or several times, in a biomass material recycling process, such as materials from polymer depolymerization or pyrolysis oil processing.

According to the invention, the “at least partially renewable and non-fossil” quality of a compound means a bio-sourced carbon content preferably between 5 wt % and 100 wt % relative to the total carbon weight of said compound.

In the context of the invention, the ASTM D6866-21 standard Method B is used to characterize the bio-sourced nature of a chemical compound and to determine the bio-sourced carbon content of said compound. The value is expressed as a weight percentage (wt %) of bio-sourced carbon relative to the total carbon weight in said compound.

The ASTM D6866-21 standard is a test method that teaches how to experimentally measure the bio-sourced carbon content of solids, liquids and gaseous samples by radiocarbon analysis.

This standard primarily uses Accelerator Mass Spectrometry (AMS) technology. This technique is used to naturally measure the radionuclides present in a sample, wherein the atoms are ionized, then accelerated to high energies, then separated, and individually counted in Faraday cups. This high-energy separation is extremely effective at filtering out isobaric interference, so that AMS is able to accurately measure abundances of carbon-14 relative to carbon-12 (14C/12C) to an accuracy of 1.10-15.

The ASTM D6866-21 standard Method B uses AMS and IRMS (Isotope Ratio Mass Spectroscopy). The test method allows to directly differentiate contemporary carbon-based carbon atoms from fossil-based carbon atoms. A measure of the carbon-14 to carbon-12 or carbon-14 to carbon-13 content of a product is determined against a modern carbon-based reference material accepted by the radiocarbon dating community such as the NIST's Standard Reference Material (SRM) 4990C (oxalic acid).

The sample preparation method is described in the standard and does not require any special comment as it is a commonly used procedure.

Analysis, interpretation and reporting of results are described below. Isotope ratios of carbon-14 to carbon-12 content or carbon-14 to carbon-13 content are measured using AMS. Isotope ratios of carbon-14 to carbon-12 content or carbon-14 to carbon-13 content are determined relative to a standard traceable via the NIST SRM 4990C modern reference standard. The “fraction of modern” (fM) represents the amount of carbon-14 in the tested product relative to the modern standard. It is most often referred to as percent modern carbon (pMC), the percentage equivalent to fM (e.g. fM 1=100 pMC).

All pMC values obtained from radiocarbon analyses must be corrected for isotopic fractionation using a given stable isotope. The correction should be made using the carbon-14 to carbon-13 values determined directly using the AMS where possible. If this is not possible, the correction should be made using the delta 13C (813C) measured by IRMS, CRDS (Cavity Ring Down Spectroscopy) or any other equivalent technology that can provide accuracy to within plus or minus 0.3 per thousand.

“Zero pMC” represents the total absence of measurable 14C in a material above the background signals, thus indicating a fossil (e.g. petroleum-based) carbon source. A value of 100 pMC indicates a fully “modern” carbon source. A pMC value between 0 and 100 indicates a proportion of carbon derived from a fossil source relative to a “modern” source.

The pMC may be higher than 100% due to the persistent, but diminishing, effects of 14C injection into the atmosphere caused by atmospheric nuclear testing programmes. The pMC values need to be adjusted by an atmospheric correction factor (REF) to obtain the actual bio-sourced content of the sample.

The correction factor is based on the excess 14C activity in the atmosphere at the time of testing. A REF value of 102 pMC was determined for 2015 based on CO2 measurements in the air in a rural area of the Netherlands (Lutjewad, Groningen). The first version of this standard (ASTM D6866-04) in 2004 had referenced a value of 107.5 pMC, while the later version ASTM D6866-10 (2010) had referenced a value of 105 pMC. These data points represent a drop of 0.5 pMC per year. Consequently, on 2 January of each year, the values in Table 1 below were used as REF value until 2019, reflecting the same decrease of 0.5 pMC per year. The REF values (pMC) for 2020 and 2021 have been determined to be 100.0 based on continuous measurements in the Netherlands (Lutjewad, Groningen) until 2019. References for reporting carbon isotope ratio data are provided below for 14C and 13C, respectively Roessler, N., Valenta, R. J., and van Cauter, S., “Time-resolved Liquid Scintillation Counting”, Liquid Scintillation Counting and Organic Scintillators, Ross, H., Noakes, J. E., and Spaulding, J. D., Eds., Lewis Publishers, Chelsea, M I, 1991, pp. 501-511. Allison, C. E., Francy, R. J., and Meijer, H. A. J., “Reference and Intercomparison Materials for Stable Isotopes of Light Elements”, International Atomic Energy Agency, Vienna, Austria, IAEATECHDOC-825, 1995.

The percentage of the bio-sourced carbon content is calculated by dividing pMC by REF and multiplying the result by 100. For example, [102 (pMC)/102 (REF)]×100=100% bio-sourced carbon. The results are indicated as a weight percentage (wt %) of bio-sourced carbon relative to the total carbon weight in said compound.

TABLE 1
Reference of percentage
of modern carbon (pMC)

| REF year | pMC |
|---|---|
| 2015 | 102.0 |
| 2016 | 101.5 |
| 2017 | 101.0 |
| 2018 | 100.5 |
| 2019 | 100.0 |
| 2020 | 100.0 |
| 2021 | 100.0 |

In the context of the invention, the term “segregated” means a material stream that is distinctive and distinguishable from other material streams in a value chain (e.g. in a product manufacturing method), and thus considered to belong to a set of materials having an equivalent nature, such that the same origin of the material, or its manufacture according to the same standard or norm, can be tracked and guaranteed throughout this value chain.

For example, this may be the case of a chemist buying 100% bio-sourced acrylonitrile exclusively from a single supplier who guarantees the 100% bio-sourced origin of the acrylonitrile delivered, and said chemist processing this 100% bio-sourced acrylonitrile separately from other potential acrylonitrile sources to produce a chemical compound. If the chemical compound produced is made solely from said 100% bio-sourced acrylonitrile, then the chemical compound is 100% bio-sourced.

In the context of the invention, the term “non-segregated”, in contrast to the term “segregated”, is understood to mean a material stream that cannot be differentiated from other material streams in a value chain.

In order to better understand this notion of segregation, it is useful to recall some basics about the circular economy and its practical application in methods, especially chemical transformation.

According to the French Environment and Energy Management Agency (ADEME), the circular economy can be defined as an economic system of trade and production which, at all stages of the life cycle of products (goods and services), seeks to increase efficiency in the use of resources and to reduce the environmental impact while developing the well-being of individuals. In other words, it is an economic system devoted to efficiency and sustainability that minimizes waste by optimizing value generated by resources. It relies heavily on a variety of conservation and recycling practices in order to break away from the current more linear “take-make-dispose” approach.

In the field of chemistry, which is the science of transforming one substance into another, this translates into reusing material that has already been used to make a product. Theoretically, all chemicals can be isolated and therefore recycled separately from other chemicals. The reality, particularly in industry, is more complex and means that even when isolated, the compound cannot often be differentiated from the same compound originating from another source, thus complicating the traceability of the recycled material.

For this reason, various traceability models have been developed taking into account this industrial reality, thereby allowing users in the chemical industry to manage their material streams with full knowledge of the facts, and allowing end customers to understand and know in a simple way the origin of the materials used to produce an object or a commodity.

These models have been developed to build transparency and trust throughout the value chain. Ultimately, this allows end-users or customers to choose a more sustainable solution without having the ability themselves to control every aspect of the method, by knowing the proportion of a desired component (e.g. of a bio-sourced nature) in an object or commodity.

One such model is “segregation”, which we have defined earlier. Some known examples where this model applies are glass and some metals where it is possible to track material streams separately.

However, chemicals are often used in complex combinations, and separate cycles are very often difficult to implement, especially due to prohibitive costs and highly complex stream management, such that the “segregation” model is not always applicable.

Consequently, when it is not possible to differentiate between material streams, other models are applied, which are grouped together under the term “non-segregated” and which entail, for example, taking into account the proportion of a specific stream relative to other streams, without physically separating the streams. One example is the Mass Balance Approach.

The Mass Balance Approach involves accurately tracking the proportion of a category (e.g. “recycled”) relative to a whole in a production system in order to guarantee, on the basis of an auditable account ledger, a proportionate and appropriate allocation of the content of that category in a finished product.

For example, this may be the case of a chemist buying 50% bio-sourced acrylonitrile from a supplier who guarantees, according to the mass or weight balance approach, that in the acrylonitrile delivered, 50% of the acrylonitrile has a bio-sourced origin, and de facto 50% is not of bio-sourced origin, and the use by said chemist of this 50% bio-sourced acrylonitrile with another stream of 0% bio-sourced acrylonitrile, the two streams not being identifiable at some point during the production process, due to mixing for example. If the chemical compound produced is made from 50% bio-sourced 50 wt % guaranteed acrylonitrile, and 0% bio-sourced 50 wt % acrylonitrile, the chemical compound is 25% bio-sourced.

In order to guarantee the stated “bio-sourced” figures, for example, and to encourage the use of recycled raw materials in producing new products, a set of globally shared and standardised rules (ISCC+, ISO 14020) has been developed to reliably manage material streams.

In the context of the invention, the term “recycled” is understood to mean the origin of a chemical compound derived from a method for recycling a material considered as waste, i.e. resulting from one or more transformations carried out using at least one recycling method on at least one material generally considered as waste.

The term “water-soluble polymer” is understood to mean a polymer which gives a clear aqueous solution when dissolved by stirring at 25° C. and with a concentration of 20 g·L⁻¹ in water.

### Method According to the Invention

The present invention relates to a biological method for obtaining an MO monomer comprising an ethylenic unsaturation by bioconversion of a CN compound comprising at least one nitrile function, said CN compound being at least partially renewable and non-fossil, said biological method comprising at least one enzymatic bioconversion step in the presence of a biocatalyst comprising at least one enzyme.

In the whole invention, the CN compound comprising at least one nitrile function is preferably (meth)acrylonitrile or 3-hydroxypropionitrile. Preferably, it comprises a single nitrile function.

In the whole invention, the MO monomer comprising an ethylenic unsaturation is preferably (meth)acrylamide, ammonium (meth)acrylate or (meth)acrylic acid.

In the present description, the expressions “between X and Y” and “from X to Y” include the terminals X and Y.

In the whole invention, the bio-sourced carbon content of a compound for which it is specified that it is at least partially renewable and non-fossil, or for which the bio-sourced carbon content is specified, relative to the total carbon weight in said compound, ranges from 5 wt % to 100 wt %, and preferably from 10 wt % to 100 wt %, preferably from 15 wt % to 100 wt %, preferably from 20 wt % to 100 wt %, preferably from 25 wt % to 100 wt %, preferably from 30 wt % to 100 wt %, preferably from 35 wt % to 100 wt %, preferably from 40 wt % to 100 wt %, preferably from 45 wt % to 100 wt %, preferably from 50 wt % to 100 wt %, preferably from 55 wt % to 100 wt %, preferably from 60 wt % to 100 wt %, preferably from 65 wt % to 100 wt %, preferably from 70 wt % to 100 wt %, preferably from 75 wt % to 100 wt %, preferably from 80 wt % to 100 wt %, preferably from 85 wt % to 100 wt %, preferably from 90 wt % to 100 wt %, preferably from 95 wt % to 100 wt %, preferably from 97 wt % to 100 wt %, preferably from 99 wt % to 100 wt %, wherein the bio-sourced carbon content is measured according to ASTM D6866-21 Method B.

In the invention and in the various embodiments described hereinafter, the CN compound comprising at least one nitrile function preferably has a bio-sourced carbon content of between 5 wt % and 100 wt % relative to the total carbon weight in said (meth)acrylonitrile, the bio-sourced carbon content being measured according to ASTM D6866-21 Method B.

In the invention and in the various embodiments described hereinafter, the (meth)acrylonitrile preferably has a bio-sourced carbon content of between 5 wt % and 100 wt % relative to the total carbon weight in said (meth)acrylonitrile, the bio-sourced carbon content being measured according to ASTM D6866-21 Method B.

In the invention and in the various embodiments described hereinafter, the 3-hydroxypriopionitrile preferably has a bio-sourced carbon content of between 5 wt % and 100 wt % relative to the total carbon weight in said 3-hydroxypriopionitrile, the bio-sourced carbon content being measured according to ASTM D6866-21 Method B.

In the invention and in the various embodiments described hereinafter, the MO monomer comprising an ethylenic unsaturation obtained according to a method of the invention has a bio-sourced carbon content of between 5 wt % and 100 wt % relative to the total carbon weight in said monomer, the bio-sourced carbon content being measured according to ASTM D6866-21 Method B.

In the invention and in the various embodiments described hereinafter, the CN compound comprising at last one nitrile function, preferably (meth)acrylonitrile or 3-hydroxypriopionitrile, are preferably totally renewable and non-fossil. And in the same manner, the monomer comprising an ethylenic unsaturation according to the invention is preferably totally renewable and non-fossil.

The CN compound comprising at least one nitrile function may be non-segregated, partially segregated or totally segregated.

Where the CN compound comprising at least one nitrile function is totally renewable and non-fossil, it may be either:
a) Totally of recycled origin and
a)1) Or totally segregated;
a)2) Or partially segregated;
a)3) Or non-segregated;
b) Or partially of recycled origin and
b)1) Or totally segregated;
b)2) Or partially segregated;
b)3) Or non-segregated;
c) Or totally of non-recycled origin and
c)1) Or totally segregated;
c)2) Or partially segregated;
c)3) Or non-segregated.

In these various embodiments, where the CN compound comprising at least one nitrile function is partially segregated, the weight ratio between the “segregated” part and the “non-segregated” part is preferably between 99:1 and 10:90, preferably between 99:1 and 30:70, or more preferably between 99:1 and 50:50.

Among these various embodiments, preference is given to the three a) embodiments, the three b) embodiments, and embodiment c)1). Among these embodiments, much greater preference is given to embodiments a)1), a)2), b)1), b)2) and c)1). The two most preferred embodiments are a)1) and b)1).

The industrial reality is such that it is not always possible to obtain industrial quantities of CN compound comprising at least one nitrile function that is bio-sourced, totally recycled and/or segregated or highly recycled and segregated. Hence, the above preferences may be more difficult to implement at the moment. From a practical standpoint, embodiments a)3), b)3), and c) are currently implemented more easily and on a larger scale. With techniques evolving quickly towards the circular economy, there is no doubt that the already applicable preferred modes will soon be applicable on a very large scale. Where the CN compound comprising at least one nitrile function is partially renewable and non-fossil, a distinction is made between the renewable part (bio-sourced) and the non-bio-sourced part. Obviously, each of these parts can be according to the same embodiments a), b) and c) described hereinabove.

As concerns the bio-sourced part of the partially bio-sourced compound, the same preferences apply as in the case where the compound is fully bio-sourced.

However, as concerns the non-bio-sourced part of the partially bio-sourced compound, it is even more preferable to have as large a recycled component as possible for a circular economy approach. Hence, in this case, preference is given to embodiments a)1), a)2), b)1), b)2), particularly a)1) and b)1).

In the invention and in the various embodiments described hereinafter, bio-acrylonitrile can be obtained by mixing glycerol, ammonia and oxygen in the gas phase in the presence of an acid catalyst, with the whole mixture being heated to high temperature to trigger the ammoxidation reaction. Alternatively, bio-acrylonitrile can be obtained by ammoxidation of bio-propylene with ammonia. Bio-propylene may be derived from a bacterial fermentation reaction of glucose, as described in Example 8 of WO 2014/086780. Lastly, in a preferred embodiment, bio-propylene is obtained by steam cracking of bio-naphtha, the latter being derived from vegetable oil as described in WO 2014/111598. In an alternative embodiment, bio-propylene is derived using a recycling method.

In an alternative embodiment, bio-propylene is derived using a pyrolysis oil processing method. This pyrolysis oil can be derived from recycling used plastics (e.g. polyesters, polypropylenes, polystyrenes, polyethylene terephthalates) and/or biomass, such as forestry residues (tall oil) and/or agricultural material. Bio-propylene can also be obtained by direct depolymerization of polypropylene.

Bio-3-hydroxypropionitrile can be obtained either directly from renewable resources or indirectly from 3-hydroxypropionic acid, itself obtained from renewable resources.

In a particular embodiment, (meth)acrylonitrile is obtained using a recycling method.

In this particular embodiment, acrylonitrile and 3-hydroxypropionitrile are obtained using a recycling method, such as from polymer depolymerization or by manufacturing from pyrolysis oil, the latter resulting from high-temperature, anaerobic combustion of used plastic waste. Thus, materials considered as waste can be used as a source to produce recycled acrylonitrile, which in turn can be used as raw material to manufacture the invention's monomer. Since the monomer according to the invention is derived using a recycling method, the polymer according to the invention hereinafter described can cater to the virtuous circle of the circular economy.

The various embodiments of the method are described hereinafter.

In a first preferred embodiment according to the invention, the MO monomer comprising an ethylenic unsaturation is acrylamide or methacrylamide.

In a first variant according to the first embodiment, the invention thus relates to a biological method for obtaining a (meth)acrylamide by bioconversion of (meth)acrylonitrile that is at least partially renewable and non-fossil, said biological method comprising at least one step of enzymatic hydrolysis of said (meth)acrylonitrile in the presence of a biocatalyst comprising at least one nitrile hydratase enzyme.

In the invention and in the various embodiments described hereinafter, the bio-(meth)acrylonitrile can be obtained either from bio-propylene or from bio-3-hydroxypropionitrile. “Bio-(meth)acrylonitrile” means bio-sourced-(meth)acrylonitrile.

In the invention and in the various embodiments described hereinafter, a biological method is understood to mean a method comprising at least one enzymatic bioconversion step in the presence of a biocatalyst comprising at least one enzyme, preferably a method comprising at least two enzymatic bioconversion steps in the presence of a biocatalyst comprising at least one enzyme.

In a second variant according to the first embodiment, the invention relates to a biological method for obtaining an acrylamide from 3-hydroxypropionitrile that is at least partially renewable and non-fossil, said process comprising at least one enzymatic bioconversion step.

In a first sub-variant of this first embodiment, 3-hydroxypropionitrile is converted into 3-hydroxypropionamide by enzymatic bioconversion in the presence of a biocatalyst comprising at least one nitrile hydratase enzyme, said 3-hydroxypropionamide then being converted into acrylamide.

In a second sub-variant according to the first embodiment, 3-hydroxypropionitrile is converted into acrylonitrile, said acrylonitrile then being converted into acrylamide according to a biological method comprising at least one step of enzymatic hydrolysis of said acrylonitrile in the presence of a biocatalyst comprising at least one nitrile hydratase enzyme.

In the whole invention, the nitrile hydratase enzyme is preferably synthesized by a microorganism of the type Bacillus, Bacteridium, Micrococcus, Brevibacterium, Corynebacterium, Pseudomonas, Acinetobacter, Xanthobacter, Streptomyces, Rhizobium, Klebsiella, Enterobacter, Erwinia, Aeromonas, Citrobacter, Achromobacter, Agrobacterium, Pseudonocardia, Rhodococcus, Comamonas, Saccharomyces, Dietzia, Clostridium, Lactobacillus, Escherichia, Agrobacterium, Mycobacterium, Methylophilus, Propionibacterium, Actinobacillus, Megasphaera, Aspergillus, Candida, or Fusarium, preferably Rhodococcus rhodochrous, and more preferably Rhodococcus rhodochrous J1.

In a second preferred embodiment according to the invention, the MO monomer comprising an ethylenic unsaturation is an acrylate or methacrylate salt.

In a first variant of the second embodiment, the (meth)acrylate salt is obtained directly from (meth)acrylonitrile that is at least partially renewable and non-fossil.

The invention therefore relates to a biological method for obtaining a (meth)acrylate salt by bioconversion of (meth)acrylonitrile that is at least partially renewable and non-fossil, said biological method comprising at least one step of enzymatic hydrolysis of said (meth)acrylonitrile in the presence of a biocatalyst comprising at least one nitrilase enzyme.

In the whole invention, the nitrilase enzyme is preferably synthesized by a microorganism of the type Bacillus, Bacteridium, Micrococcus, Brevibacterium, Corynebacterium, Pseudomonas, Acinetobacter, Xanthobacter, Streptomyces, Rhizobium, Klebsiella, Enterobacter, Erwinia, Aeromonas, Citrobacter, Achromobacter, Agrobacterium, Pseudonocardia, Rhodococcus, Comamonas, Saccharomyces, Dietzia, Clostridium, Lactobacillus, Escherichia, Agrobacterium, Mycobacterium, Methylophilus, Propionibacterium, Actinobacillus, Megasphaera, Aspergillus, Candida, or Fusarium, preferably Rhodococcus rhodochrous.

In a second variant of the second embodiment, the (meth)acrylate salt is obtained from (meth)acrylamide that is at least partially renewable and non-fossil, itself obtained from (meth)acrylonitrile that is at least partially renewable and non-fossil.

The invention therefore relates to a biological method for obtaining a (meth)acrylate salt by bioconversion of (meth)acrylamide that is at least partially renewable and non-fossil according to a biological method comprising at least one step of enzymatic hydrolysis of said (meth)acrylamide in the presence of a biocatalyst comprising at least one amidase enzyme, said (meth)acrylamide being obtained by bioconversion of (meth)acrylonitrile that is at least partially renewable and non-fossil according to a biological method comprising at least one step of enzymatic hydrolysis of said (meth)acrylonitrile in the presence of a biocatalyst comprising at least one nitrile hydratase enzyme.

In the whole invention, the amidase enzyme is preferably synthesized by the following microorganisms: Rhodococcus Erythropolis, Pseudomonas methylotropha, Rhodococcus rhodochrous or Comamonas testosteroni, and more preferably Rhodococcus rhodochrous.

In this second embodiment, the salt obtained is generally an ammonium acrylate or ammonium methacrylate. The method according to the invention may comprise a subsequent step wherein the (meth)acrylate salt is converted into (meth)acrylic acid or another (meth)acrylate salt wherein the ammonium cation is replaced by another cation, such as an alkali metal, an alkaline earth metal, preferably into sodium bio-(meth)acrylate.

In a third preferred embodiment according to the invention, the MO monomer comprising an ethylenic unsaturation is acrylic acid or methacrylic acid.

The invention therefore relates to a biological method for obtaining (meth)acrylic acid from 3-hydroxypropionitrile that is at least partially renewable and non-fossil, said biological method comprising at least one enzymatic bioconversion step in the presence of a biocatalyst comprising at least one enzyme.

In a first variant of the third embodiment, the at least partially renewable and non-fossil 3-hydroxypropionitrile is converted into 3-hydroxypropionamide by enzymatic bioconversion in the presence of a biocatalyst comprising at least one nitrile hydratase enzyme, said 3-hydroxypropionamide is then converted into a 3-hydroxypropionic acid salt by enzymatic bioconversion in the presence of a biocatalyst comprising at least one amidase enzyme, said 3-hydroxypropionic acid salt is then converted into 3-hydroxypropionic acid, and lastly, said 3-hydroxypropionic acid is converted into acrylic acid.

In a second variant of the third embodiment, the at least partially renewable and non-fossil 3-hydroxypropionitrile is converted into a 3-hydroxypropionic acid salt by enzymatic bioconversion in the presence of a biocatalyst comprising at least one nitrilase enzyme, said 3-hydroxypropionic acid salt is then converted into 3-hydroxypropionic acid, and lastly, said 3-hydroxypropionic acid is converted into acrylic acid.

In a third variant of the third embodiment, the at least partially renewable and non-fossil 3-hydroxypropionitrile is converted into 3-hydroxypropionamide by enzymatic bioconversion in the presence of a biocatalyst comprising at least one nitrile hydratase enzyme, said 3-hydroxypropionamide is then converted into a 3-hydroxypropionic acid salt by enzymatic bioconversion in the presence of a biocatalyst comprising at least one amidase enzyme, said 3-hydroxypropionic acid salt is then converted into acrylate salt, and lastly, said acrylate salt is converted into acrylic acid.

In a fourth variant of the third embodiment, the at least partially renewable and non-fossil 3-hydroxypropionitrile is converted into a 3-hydroxypropionic acid salt by enzymatic bioconversion in the presence of a biocatalyst comprising at least one nitrilase enzyme, said 3-hydroxypropionic acid salt is then converted into acrylate salt, and lastly, said acrylate salt is converted into acrylic acid.

The acrylic acid can then be converted into acrylate salt.

In a particular embodiment applicable to the various methods described in the invention, the CN compound comprising at least one nitrile function, preferably (meth)acrylonitrile or 3-hydroxypropionitrile, used in the method is derived from a recycling process.

In this particular embodiment, the method according to the invention comprises the following steps:
Recycling at least one material that is at least partially renewable and non-fossil in order to obtain a compound comprising at least one nitrile function, preferably (meth)acrylonitrile or 3-hydroxypropionitrile;
Converting said compound comprising at least one nitrile function, preferably (meth)acrylonitrile or 3-hydroxypropionitrile, into (meth)acrylamide or into a (meth)acrylate salt or into (meth)acrylic acid according to one of the previously described methods, said methods comprising at least one enzymatic bioconversion step in the presence of a biocatalyst comprising at least one enzyme.

In this particular embodiment, the method according to the invention preferably comprises the following steps:
Recycling at least one material that is least partially renewable and non-petrochemical in order to obtain (meth)acrylonitrile;
Hydrolyzing said (meth)acrylonitrile with at least one nitrilase enzyme in order to obtain an ammonium (meth)acrylate, or hydrolyzing said (meth)acrylonitrile with at least one nitrile hydratase enzyme in order to obtain a (meth)acrylamide, or hydrolyzing said (meth)acrylonitrile with at least one nitrile hydratase enzyme in order to obtain a (meth)acrylamide, and then hydrolyzing the (meth)acrylamide obtained with at least one amidase enzyme in order to obtain an ammonium (meth)acrylate.

The recycling rate is the weight ratio of the recycled material to the total material.

In this particular embodiment, the part obtained from recycling is preferably totally “segregated”, i.e. is obtained from a separate pipeline and is treated in a separate manner. In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50.

The biological method for obtaining an MO monomer comprising en ethylenic unsaturation by bioconversion of (meth)acrylonitrile that is at least partially renewable and non-fossil comprises at least one step of enzymatic hydrolysis of said (meth)acrylonitrile in the presence of a biocatalyst comprising at least one enzyme. The bioconversion may be carried out in an aqueous medium, using water as solvent and reagent. As concerns the steps and conditions of the method, the person skilled in the art may refer to the already established knowledge. In particular, he/she can consult established knowledge on methods for bioconverting (meth)acrylonitrile into (meth)acrylamide, and refer to the following documents, for example: WO 03/066800, EP 2716754 or EP 2719760.

### Monomer According to the Invention

The invention relates to an MO monomer comprising an ethylenic unsaturation obtained according to one of the previously described methods. The MO monomer comprising an ethylenic unsaturation is preferably bio-(meth)acrylamide, bio-ammonium (meth)acrylate or bio-(meth)acrylic acid. The monomer is obtained according to a biological method comprising at least one enzymatic bioconversion step in the presence of a biocatalyst comprising at least one enzyme. The same embodiments and preferences developed in the “methods” section apply to this section describing the monomer.

The invention relates to an MO monomer comprising at least an ethylenic unsaturation obtained by bioconversion of a CN compound comprising at least one nitrile function said CN compound being at least partially renewable and non-fossil, said bioconversion comprising at least an enzymatic bioconversion step in the presence of a biocatalyst comprising at least one enzyme.

In the invention and in the various embodiments described hereinafter, the CN compound comprising at least one nitrile function preferably has a bio-sourced carbon content of between 5 wt % and 100 wt % relative to the total carbon weight in said compound, the bio-sourced carbon content being measured according to ASTM D6866-21 Method B.

In the whole invention, the CN compound comprising at least one nitrile function is preferably (meth)acrylonitrile or 3-hydroxypropionitrile. Preferably, it comprises a single nitrile function.

In the whole invention, the MO monomer comprising an ethylenic unsaturation is preferably (meth)acrylamide, ammonium (meth)acrylate or (meth)acrylic acid.

In the invention and in the various embodiments described hereinafter, the MO monomer comprising an ethylenic unsaturation obtained preferably has a bio-sourced carbon content of between 5 wt % and 100 wt % relative to the total carbon weight in said monomer, the bio-sourced carbon content being measured according to ASTM D6866-21 Method B.

In the invention and in the various embodiments described hereinafter, the CN compound comprising at last one nitrile function is preferably totally renewable and non-fossil. And in the same manner, the MO monomer comprising an ethylenic unsaturation according to the invention is preferably totally renewable and non-fossil.

The various embodiments of the monomer are described hereinafter.

In a first preferred embodiment according to the invention, the MO monomer comprising an ethylenic unsaturation is acrylamide or methacrylamide.

The invention therefore relates to a bio-(meth)acrylamide obtained by bioconversion of (meth)acrylonitrile that is at least partially renewable and non-fossil, said bioconversion comprising at least one step of enzymatic hydrolysis of said (meth)acrylonitrile in the presence of a biocatalyst comprising at least one nitrile hydratase enzyme.

Preferably, the nitrile hydratase enzyme is synthesized by one of the previously mentioned microorganisms.

In the whole invention, bio-(meth)acrylamide or, more generally, bio-sourced-monomer, is understood to mean a (meth)acrylamide monomer or a monomer that is at least partially, preferably totally, derived from biomass or from synthesis gas (syngas), i.e., resulting from one or more chemical transformations carried out on one or more natural, and by contrast non-fossil, raw materials. The bio-(meth)acrylamide may also be referred to as bio-sourced or bio-resourced (meth)acrylamide.

In a second preferred embodiment according to the invention, the MO monomer comprising an ethylenic unsaturation is an acrylate or methacrylate salt.

In a first variant of the second embodiment, the (meth)acrylate salt is obtained directly from (meth)acrylonitrile that is at least partially renewable and non-fossil.

The invention therefore relates to a bio-(meth)acrylate salt obtained by bioconversion of (meth)acrylonitrile that is at least partially renewable and non-fossil, said bioconversion comprising at least one step of enzymatic hydrolysis of said (meth)acrylonitrile in the presence of a biocatalyst comprising at least one nitrilase enzyme.

In this first variant of the second embodiment, the enzyme contained in the biocatalyst is a nitrilase preferentially synthesized by one of the previously mentioned microorganisms.

In a second variant of the second embodiment, the bio-(meth)acrylate salt is obtained from (meth)acrylamide that is at least partially renewable and non-fossil, itself obtained from (meth)acrylonitrile that is at least partially renewable and non-fossil.

The invention therefore relates to a bio-(meth)acrylate salt obtained by bioconversion of (meth)acrylamide that is at least partially renewable and non-fossil, said bioconversion comprising at least one step of enzymatic hydrolysis of said (meth)acrylamide in the presence of a biocatalyst comprising at least one amidase enzyme, said (meth)acrylamide being obtained by bioconversion of (meth)acrylonitrile that is at least partially renewable and non-fossil, said bioconversion comprising at least one step of enzymatic hydrolysis of said (meth)acrylonitrile in the presence of a biocatalyst comprising at least one nitrile hydratase enzyme.

In this second variant of the second embodiment, the nitrile hydratase enzyme is preferentially synthesized by one of the previously mentioned microorganisms, and the amidase enzyme is preferentially synthesized by the previously mentioned microorganisms.

In this second embodiment, the bio-salt obtained is generally a bio-ammonium (meth)acrylate.

The invention also relates to a bio-(meth)acrylate salt different from bio-ammonium (meth)acrylate, wherein the ammonium cation is replaced by another cation, such as an alkali metal, an alkaline earth metal, or preferably sodium for example.

The invention also relates to a bio-(meth)acrylic acid obtained from the bio-(meth)acrylate salt or bio-methacrylate salt according to the invention.

The CN compound comprising at least one nitrile function may be non-segregated, partially segregated or totally segregated. The same embodiments and preferences developed in the “methods” section apply to this section describing the monomer.

In a particular embodiment, the CN compound comprising at least one nitrile function may be partially or totally of recycled origin. The same embodiments and preferences developed in the “methods” section apply to this section describing the monomer.

In this particular embodiment, the monomer according to the invention is obtained by a method comprising the following steps:
Recycling at least one renewable and non-petrochemical raw material in order to obtain acrylonitrile;
Hydrolyzing said acrylonitrile with at least one nitrilase enzyme in order to obtain an ammonium acrylate, or hydrolyzing said acrylonitrile with at least one nitrile hydratase enzyme in order to obtain an acrylamide, or hydrolyzing said acrylonitrile with at least one nitrile hydratase enzyme in order to obtain an acrylamide, and then hydrolyzing the acrylamide obtained with at least one amidase enzyme in order to obtain an ammonium acrylate;
Optionally, convert the ammonium acrylate obtained into acrylic acid or another acrylate salt, preferably into sodium acrylate.

### Polymer According to the Invention

The invention relates to a polymer obtained by polymerization of at least one monomer obtained by the method according to the invention. It also relates to a polymer obtained by polymerization of at least one monomer as previously described. The same embodiments and preferences developed in the “methods” section apply to this section describing the polymer.

The polymer according to the invention offers the advantage of being partially or totally bio-sourced, and of being produced from bio-obtained monomers, i.e. obtained according to a biological method involving a biocatalyst comprising at least one enzyme. This is known as “soft chemistry”. In an alternative embodiment, acrylonitrile may also be obtained using a recycling method. Hence the polymer according to the invention can claim to participate in the virtuous circle of the circular economy.

The polymer according to the invention is preferably water-soluble or water-swellable. The polymer may also be a superabsorbent.

The polymer according to the invention may be a homopolymer or a copolymer with at least one monomer obtained according to the method according to the invention, or with at least one of the previously described monomers, and with at least one different additional monomer, the latter advantageously being chosen from at least one nonionic monomer, and/or at least one anionic monomer, and/or at least one cationic monomer, and/or at least one zwitterionic monomer, and/or at least one monomer comprising a hydrophobic grouping.

Thus, the copolymer may comprise at least a second monomer different from the first monomer according to the invention, this second monomer being chosen from nonionic monomers, anionic monomers, cationic monomers, zwitterionic monomers, monomers comprising a hydrophobic grouping, and mixtures thereof.

The nonionic monomer is preferably selected from acrylamide, methacrylamide, N-isopropylacrylamide, N,N-dimethylacrylamide, N,N-diethylacrylamide, N-methylolacrylamide, N-vinylformamide (NVF), N-vinylacetamide, N-vinylpyridine and N-vinylpyrrolidone (NVP), N-vinyl imidazole, N-vinyl succinimide, acryloyl morpholine (ACMO), acryloyl chloride, glycidyl methacrylate, glyceryl methacrylate, and diacetone acrylamide.

The anionic monomer is preferably chosen from acrylic acid, methacrylic acid, itaconic acid, crotonic acid, maleic acid, fumaric acid, acrylamido undecanoic acid, 3-acrylamido 3-methylbutanoic acid, maleic anhydride, 2-acrylamido-2-methylpropane sulfonic acid (ATBS), vinylsulfonic acid, vinylphosphonic acid, allylsulfonic acid, methallylsulfonic acid, 2-sulfoethylmethacrylate, sulfopropylmethacrylate, sulfopropylacrylate, allylphosphonic acid, styrene sulfonic acid, 2-acrylamido-2-methylpropane disulfonic acid, and the water-soluble salts of these monomers, such as their alkali metal, alkaline earth metal or ammonium salts. It is preferably acrylic acid (and/or a salt thereof), and/or ATBS (and/or a salt thereof).

The cationic monomer is preferably chosen from quaternized dimethylaminoethyl acrylate (ADAME), quaternized dimethylaminoethyl methacrylate (MADAME), dimethyldiallylammonium chloride (DADMAC), acrylamido propyltrimethyl ammonium chloride (APTAC), and methacrylamido propyltrimethyl ammonium chloride (MAPTAC).

The zwitterionic monomer can be a derivative of a vinyl-type unit, particularly acrylamide, acrylic, allylic or maleic, this monomer having an amine or ammonium function (advantageously quaternary) and an acid function of the carboxylic (or carboxylate), sulfonic (or sulfonate) or phosphoric (or phosphate) type.

Monomers having a hydrophobic character can also be used in preparation of the polymer. Preferably, they are chosen from the group composed of esters of (meth)acrylic acid having an alkyl, arylalkyl, propoxylated, ethoxylated or ethoxylated and propoxylated chain; derivatives of (meth)acrylamide having an alkyl, arylalkyl, propoxylated, ethoxylated, ethoxylated and propoxylated chain, or dialkyl; alkyl aryl sulfonates, or of mono- or di-substituted amides of (meth)acrylamide having a propoxylated, ethoxylated, or ethoxylated and propoxylated alkyl, arylalkyl chain; derivatives of (meth)acrylamide having a propoxylated, ethoxylated, ethoxylated and propoxylated alkyl, arylalkyl, or dialkyl chain; alkyl aryl sulfonates

Each of these monomers may also be bio-sourced.

According to the invention, the polymer may have a linear, branched, star, comb, dendritic or block structure. These structures can be obtained by selecting the initiator, the transfer agent, the polymerization technique such as controlled radical polymerization referred to as RAFT (reversible addition-fragmentation chain transfer), NMP (Nitroxide Mediated Polymerization) or ATRP (Atom Transfer Radical Polymerization), incorporation of structural monomers, the concentration.

According to the invention, the polymer is advantageously linear and structured. A structured polymer refers to a non-linear polymer with side chains so as to obtain, when this polymer is dissolved in water, a pronounced state of entanglement leading to very substantial low gradient viscosities. The invention's polymer may also be cross-linked.

Additionally, the polymer according to the invention polymer may be structured:
By at least one structuring agent, which may be chosen from the group comprising polyethylenically unsaturated monomers (having at least two unsaturated functions), such as vinyl functions for example, particularly allyl, acrylic and epoxy functions, and one may mention, for example, methylene bis acrylamide (MBA), triallyamine, or tetraallylammonium chloride or 1,2 dihydroxyethylene bis-(N-acrylamide), and/or
By macroinitiators, such as polyperoxides, polyazoids and polytransfer agents, such as polymeric (co)polymers, and polyols, and/or
Functionalized polysaccharides.

The amount of branching/cross-linking agent in the monomer mixture is advantageously less than 4 wt % relative to the monomer content (weight), more advantageously less than 1%, and even more advantageously less than 0.5%. According to a particular embodiment, it may be at least equal to 0.00001 wt % relative to the monomer content.

In a particular embodiment, the polymer according to the invention may be a semi-synthetic and thus semi-natural polymer. In this embodiment, the polymer may be synthesized by copolymerization by total or partial grafting of at least one monomer according to the invention, and at least one natural compound, said natural compound being preferably chosen from starches and their derivatives, polysaccharides and their derivatives, fibers, vegetable gums, animal gums or algal gums, and modified versions thereof. For example, vegetable gums can include guar gum, gum arabic, locust bean gum, gum tragacanth, guanidinium gum, cyanine gum, tara gum, Cassia gum, xanthan gum, ghatti gum, karaya gum, gellan gum, Cyamopsis tetragonoloba gum, soy gum, or beta-glucan or dammar. The natural compound can also be gelatin, casein, or chitosan. For example, algal gum can include sodium alginate or its acid, agar-agar, or carrageenan.

Polymerization is generally carried out, without this being limiting, by copolymerization or by grafting. The person skilled in the art will be able to refer to current general knowledge in the field of semi-natural polymers.

The invention also relates to a composition comprising at least one polymer according to the invention and at least one natural polymer, said natural polymer being preferably chosen from the previously described natural polymers. The weight ratio between the synthetic polymer and the natural polymer is generally between 90:10 and 10:90. The composition may be in liquid, inverse emulsion or powder form.

In general, the polymer does not require development of a particular polymerization method. Indeed, it can be obtained according to all the polymerization techniques well known to the person skilled in the art. In particular, it can be solution polymerization; gel polymerization; precipitation polymerization; emulsion polymerization (aqueous or inverse); suspension polymerization; reactive extrusion polymerization; water-in-water polymerization; or micellar polymerization.

Polymerization is generally free radical polymerization preferably by inverse emulsion polymerization or gel polymerization. Free radical polymerization includes free radical polymerization using UV, azo, redox or thermal initiators as well as controlled radical polymerization (CRP) techniques or matrix polymerization techniques.

The polymer according to the invention can be modified after it being obtained by polymerization. This is known as post-modification of the polymer. All known post-modifications can be applied to the polymer according to the invention, and the invention also relates to polymers obtained after said post-modifications. Among the possible post-modifications developed hereinafter, mention may be made of post-hydrolysis, post-modification by Mannich reaction, post-modification by Hoffman reaction and post-modification by glyoxalation reaction.

The polymer according to the invention can be obtained by performing a post-hydrolysis reaction on a polymer obtained by polymerization of at least one monomer obtained by the method according to the invention or at least one monomer as previously described in the “Monomer” section. Prior to post-hydrolysis, the polymer comprises acrylamide or methacrylamide monomer units, for example. The polymer may also further comprise monomeric units of N-Vinylformamide. More specifically, post-hydrolysis involves reaction of hydrolyzable functional groups of advantageously non-ionic monomeric units, more advantageously amide or ester functions, with a hydrolysis agent. This hydrolysis agent may be an enzyme, an ion exchange resin, an alkali metal, or a suitable acid compound. Preferably, the hydrolysis agent is a Brønsted base. Where the polymer comprises amide and/or ester monomer units, the post-hydrolysis reaction produces carboxylate groups. Where the polymer comprises vinylformamide monomer units, the post-hydrolysis reaction produces amine groups.

The polymer according to the invention can be obtained by performing a Mannich reaction on a polymer obtained by polymerization of at least one monomer obtained by the method according to the invention or at least one monomer as previously described in the “Monomer” section. More specifically, prior to the Mannich reaction, the polymer advantageously comprises acrylamide and/or methacrylamide monomer units. The Mannich reaction is performed in aqueous solution in the presence of a dialkyl amine and a formaldehyde precursor. More advantageously, the dialkyl amine is dimethylamine and the formaldehyde precursor is formaldehyde itself. After this reaction, the polymer contains tertiary amines.

The polymer according to the invention can be obtained by performing a Hoffman reaction on a polymer obtained by polymerization of at least one monomer obtained by the method according to the invention or at least one monomer as previously described in the “Monomer” section. Prior to the Hoffman reaction, the polymer advantageously comprises acrylamide and/or methacrylamide monomer units. The so-called Hofmann degradation reaction is carried out in aqueous solution in the presence of an alkaline earth and/or alkali hydroxide and an alkaline earth and/or alkali hypohalide.

Discovered by Hofmann at the end of the nineteenth century, this reaction is used to convert an amide function into a primary amine function with one carbon atom less. The detailed reaction mechanism is presented below.

In the presence of a Brønsted base (e.g., soda), a proton is extracted from the amide.

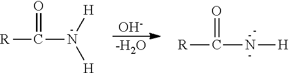

The amidate ion formed then reacts with the active chlorine (Cl₂) of the hypochlorite (e.g.: NaClO which is in equilibrium: 2 NaOH+Cl₂⇔NaClO+NaCl+H₂O) to produce an N-chloramide. The Brønsted base (e.g. NaOH) extracts a proton from the chloramide to form an anion. The anion loses a chloride ion to form a nitrene which undergoes isocyanate rearrangement.

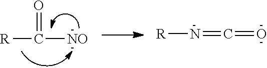

By reaction between the hydroxide ion and the isocyanate, a carbamate is formed.

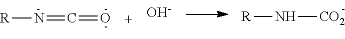

After decarboxylation (removal of CO2) from the carbamate, a primary amine is obtained.

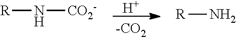

For the conversion of all or part of the amide functions of a (co)polymer comprising an amide group into an amine function, two main factors are involved (expressed in molar ratios). These are:
Alpha=(alkali and/or alkaline earth hypohalide/amide group) and
Beta=(alkali and/or alkaline earth hydroxide/alkali and/or alkaline earth hypohalide).

The polymer according to the invention can also be obtained by carrying out a glyoxalation reaction on a polymer obtained by polymerization of at least one monomer obtained by the method according to the invention or of at least one monomer as previously described in the “Monomer” section, said polymer comprising, with the glyoxalation reaction, at least one monomer unit advantageously of acrylamide or methacrylamide. More specifically, the glyoxalation reaction involves a reaction of at least one aldehyde on the polymer, thus allowing said polymer to be functionalized. Advantageously, the aldehyde may be chosen from the group comprising glyoxal, glutaraldehyde, furan dialdehyde, 2-hydroxyadipaldehyde, succinaldehyde, starch dialdehyde, 2.2 dimethoxyethanal, diepoxy compounds, and combinations thereof. Preferably, the aldehyde compound is glyoxal.

According to the invention, the polymer may be in liquid, gel or solid form when its preparation includes a drying step such as spray drying, drum drying, radiation drying, such as microwave drying, or fluid bed drying.

According to the invention, the water-soluble polymer preferably has a molecular weight between 1000 and 40 million g/mol. The polymer may be a dispersant, in which case its molecular weight is preferably between 1000 and 50,000 g/mol. The polymer may have a higher molecular weight, typically between 1 and 30 million g/mol. The molecular weight is understood as weight average molecular weight. The polymer according to the invention may also be a superabsorbent capable of absorbing from 10 to 500 times its weight in water.

The molecular weight is advantageously determined by the intrinsic viscosity of the (co)polymer. The intrinsic viscosity can be measured by methods known to the person skilled in the art and can be calculated from the reduced viscosity values for different (co)polymer concentrations by a graphical method entailing plotting the reduced viscosity values (y-axis) against the concentration (x-axis) and extrapolating the curve down to zero concentration. The intrinsic viscosity value is plotted on the y-axis or using the least squares method. The molecular weight can then be determined using the Mark-Houwink equation:

[η]=KM^{α}
[η] represents the intrinsic viscosity of the (co)polymer determined by the solution viscosity measurement method.
K represents an empirical constant.
M represents the molecular weight of the (co)polymer.
α represents the Mark-Houwink coefficient.
K and a depend on the specific (co)polymer-solvent system.

The co-monomers combined with the monomer according to the invention to obtain the polymer of the invention, are preferably at least partially, or more preferably totally renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention, and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention, and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil;
at least 1 mol %, preferably between 5 mol % and 90 mol %, more preferably between 10 mol % and 80 mol % of at least one third monomer comprising an ethylenic unsaturation, said third monomer being different from the first and the second monomers, and being at least partially renewable and non-fossil.

The polymer according to the invention may comprise four or more different monomers.

In a preferred embodiment, the second and the possible other monomers have a bio-sourced carbon content ranging between 5 wt % and 100 wt %, preferably 10 wt % and 100 wt %, relative to the total carbon weight in the related monomer, the bio-sourced carbon content being measured according to ASTM D6866-21 Method B.

In this preferred embodiment, the second and the possible other monomers are preferably chosen from an oligomer of acrylic acid, 2-acrylamido-2-methylpropane sulfonic acid (ATBS) and/or a salt thereof, N-vinylformamide (NVF), N-vinylpyrrolidone (NVP), dimethyldiallylammonium chloride (DADMAC) quaternized dimethylaminoethyl acrylate (ADAME), quaternized dimethylaminoethyl methacrylate (MADAME), a substituted acrylamide having the formula CH₂═CHCO—NR¹R², R¹ and R² being, independently of each other, a linear or branched carbon chain CₙH₂ₙ₊₁, wherein n is between 1 and 10.

In the whole invention, it will be understood that the molar percentage of the monomers (excluding any cross-linking agents) of the polymer is equal to 100%.

The CN compound comprising at least one nitrile function may be non-segregated, partially segregated or totally segregated. The same embodiments and preferences developed in the “methods” section apply to this section describing the polymer.

In a particular embodiment, the CN compound comprising at least one nitrile function may be partially or totally of recycled origin. The same embodiments and preferences developed in the “methods” section apply to this section describing the polymer.

The invention further relates to a polymer as previously described, comprising a bio-sourced carbon content of between 5 wt % and 100 wt % relative to the total carbon weight in said polymer, the bio-sourced carbon content being measured according to ASTM D6866-21 Method B.

The invention also relates to the use of at least one monomer obtained by the method according to the invention in order to synthesize a polymer.

### Using the Polymer According to the Invention

The invention also relates to the use of the polymer according to the invention in the recovery of hydrocarbons (oil and/or gas); in drilling and cementing of wells; in the stimulation of hydrocarbon wells (oil and/or gas), for example hydraulic fracturing, conformation, diversion; in the treatment of water in open, closed or semi-closed circuits; in the treatment of fermentation slurry, treatment of sludge; in paper manufacturing; in construction; in wood processing; in hydraulic composition processing (concrete, cement, mortar and aggregates); in the mining industry; in the formulation of cosmetic products; in the formulation of detergents; in textile manufacturing; in battery component manufacturing; in geothermal energy; in sanitary napkin manufacturing; or in agriculture.

The invention also relates to the use of the polymer according to the invention as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

### Method Using the Polymer According to the Invention

The present invention also relates to the various methods described hereinafter, wherein the polymers of the invention are used to improve application performance.

The invention also relates to a method for enhanced oil and/or gas recovery by sweeping a subterranean formation comprising the following steps:
a. Preparing an injection fluid from a polymer according to the invention with water or brine,
b. Injecting the injection fluid into a subterranean formation,
c. Sweeping the subterranean formation with the injected fluid,
d. Recovering an aqueous mixture of oil and/or gas.

The invention also relates to a method for hydraulic fracturing of subterranean oil and/or gas reservoirs comprising the following steps:
a. Preparing an injection fluid from a polymer according to the invention, with water or brine, and with at least one proppant,
b. Injecting said fluid into the subterranean reservoir and fracturing at least a portion thereof to recover oil and/or gas.

In the methods described hereinabove, the polymer is preferably a high molecular weight polymer (greater than 8 million daltons). It is preferably linear. It is preferably in the form of a powder, an inverse emulsion, a partially dehydrated inverse emulsion, or in the form of a “clear”, i.e. a dispersion of solid polymer particles in an aqueous or oily fluid. The powder form is preferably obtained by gel or spray drying of an inverse emulsion. It also involves a composition comprising an inverse emulsion of a polymer according to the invention and solid particles of a polymer according to the invention.

The invention also relates to a method of stimulation of a subterranean formation comprising the following steps:
a. Preparing an injection fluid from a polymer according to the invention with water or brine,
b. Injecting the injection fluid into a subterranean formation,
c. Partially or totally plugging the subterranean formation with the injected fluid, said plugging being temporary or permanent.

The invention also related a method of drilling and/or cementing a well in a subterranean formation comprising the following steps:
a. Preparing an injection fluid from a polymer according to the invention with water or brine,
b. Injecting said drilling and/or cementing fluid into the subterranean formation via the drill head in at least one step of drilling or cementing a well.

Drilling and cementing a well are two successive steps in creating a well in a subterranean formation. The first step is drilling with the drilling fluid, while the second step is cementing the well with the cementing fluid. The invention also relates to a method of injecting an intermediate fluid (“spacer fluid”) injected between the drilling fluid and the cementing fluid, said intermediate fluid comprising at least one polymer according to the invention. This intermediate fluid prevents contamination between the cementing fluid and the drilling fluid.

When drilling and cementing a well, the polymer according to the invention can be used as a fluid loss additive in well cement compositions in order to reduce fluid loss from the cement compositions to permeable formations or zones into or through which the cement compositions are pumped. In primary cementing, loss of fluid, i.e., water, to permeable formations or subterranean zones can lead to premature gelling of the cement composition, so that bridging the annular space between the permeable formation or zone and the drill string cemented therein prevents the cement composition from being placed along the entire length of the ring.

The invention also relates to a method of inerting clays in hydraulic compositions for construction purposes, said method comprising a step of adding to the hydraulic composition or one of its constituents at least one clay inerting agent, characterized in that the clay inerting agent is a polymer according to the invention.

Clays can absorb water and cause poor performance of building materials. When the polymer of the invention is used as a clay inhibitor, it allows in particular to avoid the clay swelling which may cause cracks thus weakening any building.

The hydraulic composition may be a concrete, cement, mortar or aggregate. The polymer is added to the hydraulic composition or to one of its constituents advantageously at a dosage of 2 to 200 ppm of inerting agent relative to the weight of aggregate.

In this method of inerting clays, clays include, but are not limited to, 2:1 swelling clays (such as smectite), or 1:1 swelling clays (such as kaolin) or 2:1:1 swelling clays (such as chlorite).

The term “clay” generally refers to magnesium and/or aluminum silicate, including phyllo silicates with a lamellar structure. However, in the present invention, the term “clay” also includes clays having no such structure, such as amorphous clays.

The invention also relates to a method for manufacturing a sheet of paper, cardboard or the like, whereby, before a sheet is formed, a step is performed entailing adding to a suspension of fibers, at one or more injection points, at least one polymer according to the invention. The polymer may provide dry strength or retention properties or wet strength. It may also improve paper formation, drainage and dewatering capabilities.

The method can be used successfully to manufacture packaging papers and cardboards, coating papers, sanitary and household papers, any type of paper, cardboard or the like.

The post-modified polymers described in the “Polymers” section, in particular the post-modified polymers by Hoffman reaction or by glyoxalation reaction, are particularly advantageous in methods for manufacturing paper, cardboard or the like.

Retention properties are understood to mean the capability to retain the suspended materials of the paper pulp (fibers, fines, fillers (calcium carbonate, titanium oxide), . . . ) on the forming fabric, thus in the fibrous mat that will make up the final sheet. The mode of action of the retention agents is based on the flocculation of these suspended materials in water. Indeed, the flocs formed are more easily retained on the forming sheet.

The retention of fillers involves retaining specifically the fillers (small mineral species with little affinity with cellulose). Substantial improvement of retention of fillers leads to a clarification of white water by retaining the fillers in the sheet and by increasing its grammage. It also gives the possibility to replace part of the fibers (the most expensive species in the composition of paper, cardboard or similar) with fillers (lower costs) in order to reduce manufacturing costs.

As concerns dewatering (or drainage) properties, it is the capacity of the fibrous mat to evacuate or drain the maximum amount of water so that the sheet dries as quickly as possible, in particular during manufacturing of the sheet.

These two properties (retention and drainage) being intricately linked, one depending on the other, the issue is therefore to find the best compromise between retention and drainage. Generally, the person skilled in the art refers to a retention and drainage agent because these are the same types of products used to improve these two properties.

Fibrous suspension is understood to mean thick pulp or diluted pulp which are composed of water and cellulose fibers. The thick stock, with a dry matter concentration of more than 1% or even more than 3%, is located upstream of the fan pump. The thin stock, with a dry mass concentration of generally less than 1%, is located downstream of the fan pump.

The polymer can be added to the thick stock or to the thin stock. It can be added at the level of the fan pump or the headbox. Preferably, the polymer is added before the headbox.

In the method for making paper, cardboard or the like according to the invention, the polymer according to the invention may be used alone or in combination with a secondary retention agent. Preferably, a secondary retention agent selected from organic polymers and/or inorganic microparticles is added to the fiber suspension.

This secondary retention agent added to the fibrous suspension is advantageously chosen from anionic polymers in the broad sense, which can therefore be (without being limiting) linear, branched, cross-linked, hydrophobic, associative and/or inorganic microparticles (such as bentonite, colloidal silica).

The invention also relates to a method for treating a suspension of solid particles in water resulting from mining or oil sands operations, comprising contacting said suspension with at least one polymer according to the invention. Such a method can be carried out in a thickener, which is a holding zone, generally in the form of a tube section of several meters in diameter with a conical bottom wherein the particles can settle. According to a specific embodiment, the aqueous suspension is transported by means of a pipe to a thickener, and the polymer is added to said pipe.

According to another embodiment, the polymer is added to a thickener that already contains the suspension to be treated. In a typical mineral processing operation, the suspensions are often concentrated in a thickener. This results in a higher density sludge that exits the bottom of the thickener, and an aqueous fluid released from the treated suspension (called liquor) that exits by overflow, from the top of the thickener. Generally, the addition of the polymer increases the concentration of the sludge and increases the clarity of the liquor.

According to another embodiment, the polymer is added to the particulate suspension during transport of said suspension to a deposition area. Preferably, the polymer is added in the pipe that conveys said suspension to a deposition zone. It is on this deposition area that the treated suspension is spread in preparation for dewatering and solidification. The deposition areas can be either open, such as an unconfined area of soil, or enclosed, such as a basin, cell.

An example of such treatments during transport of the suspension is spreading the suspension treated with the polymer according to the invention on the soil in preparation for dewatering and solidification and then spreading a second layer of treated suspension on top of the solidified first layer. Another example is the continuous spreading of the suspension treated with the polymer according to the invention in such a way that the treated suspension falls continuously on the suspension previously discharged in the deposition area, thus forming a mass of treated material from which water is extracted.

According to another embodiment, the water-soluble polymer is added to the suspension and a mechanical treatment is performed, such as centrifugation, pressing or filtration.

The water-soluble polymer can be added simultaneously in different stages of the suspension treatment, i.e., for example, in the pipe carrying the suspension to a thickener and in the sludge exiting the thickener which will be conveyed either to a deposition area or to a mechanical treatment device.

The invention also relates to a method for treating municipal or industrial water, comprising the introduction into said water to be treated of at least one polymer according to the invention. Effective water treatment requires the removal of dissolved compounds, and dispersed and suspended solids from the water. Generally, this treatment is enhanced by chemicals such as coagulants and flocculants. These are usually added to the water stream ahead of the separation unit, such as flotation and sedimentation.

The polymers according to the invention can be advantageously used to coagulate or flocculate suspended particles in municipal or industrial wastewater. Generally, they are used in combination with inorganic coagulants such as alum.

They can also be used advantageously to treat the sludge produced from the treatment of this wastewater. Sewage sludge (be it urban or industrial) is the main waste produced by a treatment plant from liquid effluents. Generally, sludge treatment involves dewatering it. This dewatering can be performed by centrifugation, filter press, belt press, electro-dewatering, sludge drying reed beds, solar drying. It is used to decrease sludge water concentration.

In this municipal or industrial water treatment process, the polymer according to the invention is preferably linear or branched. It is preferably in the form of a powder, an inverse emulsion or a partially dehydrated inverse emulsion. The powder form is preferably obtained by gel or spray drying from an inverse emulsion.

The invention also relates to an additive for a cosmetic, dermatological or pharmaceutical composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to cosmetic, dermatological or pharmaceutical compositions comprising at least one polymer according to the invention.

In particular, reference may be made to application FR2979821 on behalf of L'OREAL for the manufacture of such compositions and description of the other ingredients of such compositions. The said compositions may be in the form of a milk, a lotion, a gel, a cream, a gel cream, a soap, a bubble bath, a balm, a shampoo or a conditioner. The use of said compositions for the cosmetic or dermatological treatment of keratinous materials, such as the skin, scalp, eyelashes, eyebrows, nails, hair and/or mucous membranes is also an integral part of the invention. Such use comprises application of the composition to the keratinous materials, possibly followed by rinsing with water.

The invention also relates to an additive for detergent composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to detergent compositions for household or industrial use comprising at least one polymer according to the invention. In particular, reference may be made to the applicant's application WO2016020622 for the manufacture of such compositions and description of the other ingredients of such compositions.

“Detergent compositions for household or industrial use” are understood to mean compositions for cleaning various surfaces, particularly textile fibers, hard surfaces of any kind such as dishes, floors, windows, wood, metal or composite surfaces. Such compositions include, for example, detergents for washing clothes manually or in a washing machine, products for cleaning dishes manually or for dishwashers, detergent products for washing house interiors such as kitchen elements, toilets, furnishings, floors, windows, and other cleaning products for universal use.

The polymer used as an additive, e.g., thickener, for a cosmetic, dermatological, pharmaceutical, or detergent composition is preferably cross-linked. It is preferably in the form of a powder, an inverse emulsion or a partially dehydrated inverse emulsion. The powder form is preferably obtained by spray drying from an inverse emulsion.

The invention equally relates to a thickener for pigment composition used in textile printing, said thickener comprising at least one polymer according to the invention. The invention also relates to a textile fiber sizing agent, said agent comprising at least one polymer according to the invention.

The invention also relates to a process for manufacturing superabsorbent from the monomer according to the invention, a superabsorbent obtained from at least one monomer according to the invention, said superabsorbent to be used for absorbing and retaining water in agricultural applications or for absorbing aqueous liquids in sanitary napkins. For example, the superabsorbent agent is a polymer according to the invention.

The invention also relates to a method for manufacturing sanitary napkins wherein a polymer according to the invention is used, for example as a superabsorbent agent.

The invention also relates to the use of the polymer according to the invention as a battery binder. The invention also relates to a battery binder composition comprising the polymer according to the invention, an electrode material and a solvent. The invention also relates to a method for manufacturing a battery comprising making a gel comprising at least one polymer according to the invention and filling same into said battery. Mention may be made of lithium ion batteries which are used in a variety of products, including medical devices, electric cars, aircraft and, most importantly, consumer products such as laptops, cell phones and cameras.

Generally, lithium ion batteries (LIBs) include an anode, a cathode, and an electrolyte material such as an organic solvent containing a lithium salt. More specifically, the anode and cathode (collectively, the “electrodes”) are formed by mixing an electrode active material (anode or cathode) with a binder and solvent to form a paste or sludge that is then applied and dried onto a current collector, such as aluminum or copper, to form a film on the current collector. The anode and cathode are then stacked and wound before being housed in a pressurized case containing an electrolyte material, all of which together form a lithium-ion battery.

In a lithium battery, the binder plays several important roles in both mechanical and electrochemical performance. Firstly, it helps disperse the other components in the solvent during the manufacturing process (some also act as a thickener), thus allowing for even distribution. Secondly, it holds the various components together, including the active components, any conductive additives, and the current collector, ensuring that all of these parts stay in contact. Through chemical or physical interactions, the binder connects these separate components, holding them together and ensuring the mechanical integrity of the electrode without a material impact on electronic or ionic conductivity. Thirdly, it often serves as an interface between the electrode and the electrolyte. In this role, it can protect the electrode from corrosion or the electrolyte from depletion while facilitating ion transfer across this interface.

Another important point is that the binders must have a certain degree of flexibility so that they do not crack or develop defects. Brittleness can create problems during manufacturing or assembly of the battery.

Given all the roles it plays in an electrode (and in the battery as a whole), choosing a binder is critical in ensuring good battery performance.

The invention also relates to a method for manufacturing sanitary napkins wherein a polymer according to the invention is used, for example as a superabsorbent agent.

As previously described, the circular economy is an economic system devoted to efficiency and sustainability that minimizes waste by optimizing value generated by resources. It relies heavily on a variety of conservation and recycling practices in order to break away from the current more linear “take-make-dispose” approach.

Therefore, with material recycling being a major and growing concern, recycling processes are developing rapidly and enabling the production of materials that can be used to produce new compounds or objects. Recycling materials does not depend on the origin of the material and as long as it can be recycled, it is considered as a technical progress. Although the origin of the material to be recycled may be renewable and non-fossil, it may also be fossil.

Specific objects are described hereinafter.

A first specific object relates to a biological method for obtaining an MO monomer comprising an ethylenic unsaturation by bioconversion of a CN compound comprising at least one nitrile function, said biological method comprising at least one step of enzymatic hydrolysis of said CN compound in the presence of a biocatalyst comprising at least one enzyme, said CN compound derived at least partially, preferably entirely, from a process of recycling a renewable and non-fossil material, or a fossil material.

Preferably, the CN compound comprising at least one nitrile function is totally “segregated”, i.e. derived from a separate pipeline and treated separately. In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50. In an alternative embodiment, it is totally “segregated”.

A second specific object relates to an MO monomer comprising an ethylenic unsaturation obtained by bioconversion of a CN compound comprising at least one nitrile function, said bioconversion comprising at least one step of enzymatic hydrolysis of said CN compound in the presence of a biocatalyst comprising at least one enzyme, said CN compound derived at least partially, preferably entirely, from a process of recycling a renewable and non-fossil material, or a fossil material.

A third specific object relates to the (meth)acrylamide obtained by bioconversion of a CN compound comprising at least one nitrile function, said bioconversion comprising at least one step of enzymatic hydrolysis of said CN compound in the presence of a biocatalyst comprising at least one enzyme, said CN compound derived at least partially, preferably entirely, from a process of recycling a renewable and non-fossil material, or a fossil material.

A fourth specific object relates to the (meth)acrylic acid or (meth)acrylate salt obtained by bioconversion of a CN compound comprising at least one nitrile function, said bioconversion comprising at least one step of enzymatic hydrolysis of said CN compound in the presence of a biocatalyst comprising at least one enzyme, said CN compound derived at least partially, preferably entirely, from a process of recycling a renewable and non-fossil material, or a fossil material.

A polymer obtained by polymerization of at least one (meth)acrylamide monomer or (meth)acrylic acid or (meth)acrylate salt as just previously described.

A fifth specific object relates to the use of a polymer obtained by polymerization of at least one (meth)acrylamide monomer or (meth)acrylic acid or (meth)acrylate salt as just previously described, in the oil and gas recovery, in drilling and cementing of wells; in the stimulation of oil and gas wells (for example hydraulic fracturing, conformation, diversion), in the treatment of water in open, closed or semi-closed circuits, in the treatment of fermentation slurry, treatment of sludge, in paper manufacturing, in construction, in wood processing, in hydraulic composition processing (concrete, cement, mortar and aggregates), in the mining industry, in the formulation of cosmetic products, in the formulation of detergents, in textile manufacturing, in battery component manufacturing, in geothermal energy, or in agriculture.

A sixth specific object relates to the use of a polymer obtained by polymerization of at least one (meth)acrylamide monomer or (meth)acrylic acid or (meth)acrylate salt as just previously described as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

A seventh specific object relates to a polymer obtained according to a method comprising the following steps:
Recycling at least one renewable and non-fossil or fossil raw material in order to obtain (meth)acrylonitrile;
Hydrolyzing said (meth)acrylonitrile with at least one nitrilase enzyme in order to obtain an ammonium (meth)acrylate, or hydrolyzing said (meth)acrylonitrile with at least one nitrile hydratase enzyme in order to obtain a (meth)acrylamide, or hydrolyzing said (meth)acrylonitrile with at least one nitrile hydratase enzyme in order to obtain a (meth)acrylamide, and then hydrolyzing the (meth)acrylamide obtained with at least one amidase enzyme in order to obtain an ammonium (meth)acrylate;
Optionally, convert the ammonium(meth) acrylate obtained into (meth)acrylic acid or another (meth)acrylate salt, preferably into sodium (meth)acrylate;
Polymerizing the ammonium (meth)acrylate and/or (meth)acrylic acid, and/or another (meth)acrylate salt, and/or (meth)acrylamide, and/or optionally, another unsaturated monomer.

Said (meth)acrylonitrile being preferably totally “segregated”, i.e. derived from a separate pipeline and treated separately.

In an alternative embodiment, it is partially “segregated” and partially “non-segregated”. In this case, the weight ratio between the “segregated” part and the “non-segregated” part is preferably between 99:1 and 10:90, preferably between 99:1 and 30:70, more preferably between 99:1 and 50:50. In an alternative embodiment, it is totally “segregated”.

FIG. 1 inter alia shows details in a general diagram of the various ways of obtaining the monomers according to the invention.

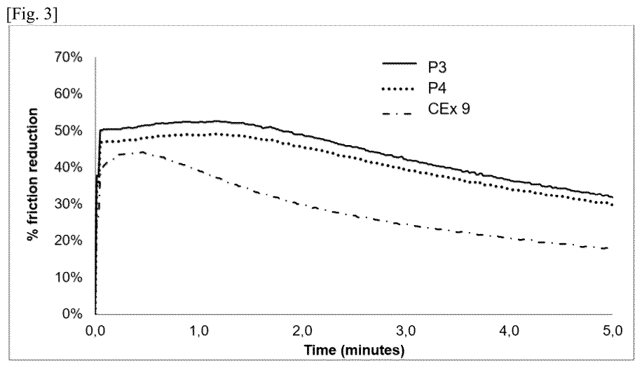

FIGS. 2 and 3 are graphs representing the percentage reduction in friction as a function of time for brines containing polymers.

### EXAMPLES

The following examples relate to the synthesizing of monomers comprising an ethylenic unsaturation by bioconversion of a bio-sourced compound comprising at least one nitrile function.

Using these examples, we can best illustrate the advantages of said invention in a clear and non-limiting manner.

### Description of the Gas Chromatographic Analysis Method for Residual Acrylonitrile

The residual acrylonitrile in the acrylamide solution is measured by gas phase chromatography; this measurement is made by a gas phase chromatograph with a flame ionization detector (AUTOSYSTEM XL type from Perkin Elmer).

The different compounds present in the sample are identified by their retention time in the column which are represented by peaks. Their concentration is calculated using the ratio of the areas of the peaks, using a calibration made from internal benchmarking standards.

For calibration, benchmarking standards are prepared with contents of 10, 50, 100, 150, 200 and 250 ppm of acrylonitrile, and with 5 wt % of an internal benchmark (methacrylamide).

The acrylamide samples to be analysed are filtered at 0.45 μm and 5 wt % of methacrylamide is added.

The retention time of acrylonitrile is 0.5 minutes, and that of methacrylamide 4.5 minutes.

The column is 1-meter-long and has a diameter of ⅛ inch (reference PORAPAK PS).

The analysis conditions are as follows:
Injector temperature: 250° C.
Oven temperature: 170° C. (isothermal).
Detector temperature: 250° C.
Carrier gas flow: 25 ml/min of nitrogen.
Injection volume: 0.5 μl.
Analysis time: 6 min.

### Description of the Liquid Chromatography Analysis Method for Residual Acrylamide

Residual acrylamide is measured by liquid phase chromatography equipped with a UV detector.

The different compounds present in the sample are identified by their retention time in the column which are represented by peaks. Their concentration is calculated from the ratio of the areas of the peaks using a calibration made from internal benchmarking standards.

Acrylamide retention time is 2.5 minutes.

The column is an Atlantis dC18 reverse phase column with a length of 150 mm, an internal diameter of 4.6 mm.

The analysis conditions are as follows:
Wavelength: 205 nm.
Injection rate: 1.0 ml/min.
Mobile phase: 85% by volume of a 20 mM/L KH2PO4 buffer at pH=3.8 and 15% methanol.
Injection volume: 10 μL.
Analysis time: 8 minutes.

### Description of the Filtration Quotient Measurement Test

The term filtration ratio is used herein to refer to a test used to determine the performance of the polymer solution under conditions approaching reservoir permeability by measuring the time taken for given volumes/concentrations of solution to pass through a filter. The FR generally compares the filterability of the polymer solution for two consecutive equivalent volumes, which indicates the tendency of the solution to clog the filter. Lower FRs indicate better performance.

The test used to determine the FR consists of measuring the times it takes for given volumes of solution containing 1000 active ppm of polymer to flow through a filter. The solution is contained in a pressurized cell at two bars of pressure and the filter is 47 mm in diameter and of defined pore size. Generally, the Fr is measured with filters having a pore size of 1.2 μm, 3 μm, 5 μm or 10 μm.

The times required to obtain 100 ml (t100 ml); 200 ml (t200 ml) and 300 ml (t300 ml) of filtrate are therefore measured and a FR is then defined, expressed by:

$$FR = \frac{{t}_{300 ml} - {t}_{200 ml}}{{t}_{200 ml} - {t}_{100 ml}}$$

Times are measured to within 0.1 seconds.

The FR thus represents the capacity of the polymer solution to clog the filter for two equivalent consecutive volumes.

### Description of the Chemical Degradation Test

The test used to determine resistance to chemical degradation consists of preparing a polymer solution at a given concentration in a given brine under aerobic conditions and bringing it into contact with a chemical contaminant such as iron or hydrogen sulphide. The viscosity of the polymer solution is measured before and after 24 h of exposure to the contaminant. The viscosity measurements are carried out under the same temperature and shear rate conditions.

The resistance to chemical degradation is quantified by the viscosity loss value expressed as a percentage and determined at maturity by:

$$Viscosity loss ( \% ) = \frac{{Viscosity}_{initial} - {Viscosity}_{at maturity}}{{Viscosity}_{initial}} \times 100$$

### Example 1: Synthesis of Acrylamide

A test set is made, adjusting the origin of acrylonitrile, its percentage of ¹⁴C. as well as the dose of enzyme used in order to carry out the examples summarized in Table 2.

The wt % of ¹⁴C is indicative of the nature of the carbon. The levels of ¹⁴C in the different acrylonitriles are measured according to the ASTM D6866-21 standard, method B. This standard makes it possible to characterize the bio-sourced nature of a chemical compound by determining the bio-sourced carbon level of said compound. A “zero pMC” represents the total absence of measurable ¹⁴C in a material, thus indicating a fossil carbon source.

The acrylonitrile of biological origin can come from the treatment of residues from the paper pulp industry (“tall oil” in English) in order to form the bio-propylene precursor before the ammoxidation process.

Alternatively, it may come from the processing of vegetable oil according to patent WO 2014/111598 or recycled cooking oil.

### Protocol:

In a 1000 mL reactor equipped with a jacket, a stirrer and a condenser are added 621.5 g of deionized water. The initial pH is adjusted to 8 with 10% sodium hydroxide.

The contents of the reactor are cooled to a temperature of 20° C. using a cryothermostat supplying the jacket of the reactor.

An enzyme, nitrile hydratase expressed by a microorganism Rhodococcus rhodochrous J1 is added to the reaction medium. The enzyme has a dry extract of 10 wt %.

373 g of acrylonitrile is continuously added to the reactor at a rate of 46.6 g per hour.

The enzymatic conversion reaction of acrylonitrile is exothermic, the reactor is cooled by the jacket using the cryothermostat, so as to maintain a temperature of between 20 and 25° C. in the reaction medium.

At the end of the addition of acrylonitrile, a ripening time of 1 hour is applied in order to convert a maximum of acrylonitrile. A sample of the reaction medium is taken for analysis by gas phase chromatography in order to determine the quantity of residual acrylonitrile.

The residual quantity of acrylonitrile must be less than 100 ppm to validate the bioconversion test of acrylonitrile to acrylamide.

TABLE 2

| | | | | Amount | Residual |
|---|---|---|---|---|---|
| | | | % | of | amount of |
| | Origin of | Purity of | weight | enzyme | acrylonitrile |
| | acrylonitrile | acrylonitrile | ¹⁴C | (mg) | (ppm) |
| CEx 1 | Fossil | 99.2% | 0 | 400 | 8,932 |
| CEx 2 | Fossil | 99.2% | 0 | 500 | 98 |
| Inv 1 | Organic (tall | 99% | 80 | 400 | 63 |
| (Invention) | oil) | | | | |
| Inv 2 | Organic (tall | 99% | 80 | 350 | 95 |
| (Invention) | oil) | | | | |
| Inv 3 | Organic (tall | 99% | 80 | 500 | 5 |
| (Invention) | oil) | | | | |
| Inv 4 | Organic (tall | 99% | 60 | 400 | 75 |
| (Invention) | oil) | | | | |
| Inv 5 | Organic | 99.1% | 70 | 400 | 70 |
| (Invention) | (vegetable oil) | | | | |
| Inv 6 | Organic | 99.1% | 100 | 400 | 10 |
| (Invention) | (vegetable oil) | | | | |
| Inv 7 | Organic (tall | 99.1% | 100 | 400 | 8 |
| (Invention) | oil) | | | | |

In table 2, the applicant observes that the renewable origin of acrylonitrile makes it possible to reduce the quantity of enzyme necessary for the reaction.

By comparing counterexample CEx 2 and example Inv 3 (same quantity of enzyme), the quantity of residual acrylonitrile is reduced by a factor close to 20.

By comparing counterexample CEx 2 and example Inv 2, the applicant notes that approximately 30% less catalyst is needed to arrive at the same residual quantity of acrylonitrile at the end of the bioconversion.

### Example 2: Bioconversion of Acrylonitrile to Acrylamide

A set of tests is carried out, adjusting the origin of the acrylonitrile, its percentage of 14° C., as well as the dose of enzyme used to carry out examples Inv 8 to Inv 14 and counterexamples CEx 3 and CEx 4, which are summarised in Table 3.

### Protocol

6 reactors are connected in cascade, with a unit volume of 1000 litres. Each is equipped with stirring and a double jacket supplied with glycol water.

The temperature of the reaction medium of each of the reactors is controlled at 20° C. Deionized water is fed to the 1st reactor at a flow rate of 380 litres per hour. Acrylonitrile is fed to the 1st reactor at a flow rate of 218 litres per hour. The second reactor is fed with acrylonitrile at a flow rate of 73 litres per hour.

A nitrile hydratase enzyme expressed by a Rhodococcus rhodochrous J1 microorganism is added to the first reactor. The enzyme has a dry extract of 10 wt %.

The carbon 14 level in the different acrylonitriles is measured according to ASTM D6866-21 method B.

The residual quantity of acrylonitrile must be less than 100 ppm to validate the bioconversion test of acrylonitrile to acrylamide.

TABLE 3

| | | | | | Residual |
|---|---|---|---|---|---|
| | | | | Enzyme | amount of |
| | | | | quantity | acrylonitrile |
| | | | % | (litre | in the last |
| | Origin of | Purity of | weight | per | reactor |
| | acrylonitrile | acrylonitrile | ¹⁴C | hour) | (ppm) |
| CEx 3 | Fossil | 99.2% | 0 | 0.27 | 2312 |
| CEx 4 | Fossil | 99.2% | 0 | 0.336 | 91 |
| Inv 8 | Organic (tall | 99% | 80 | 0.27 | 67 |
| (Invention) | oil) | | | | |
| Inv 9 | Organic (tall | 99% | 80 | 0.25 | 97 |
| (Invention) | oil) | | | | |
| Inv 10 | Organic (tall | 99% | 80 | 0.336 | 3 |
| (Invention) | oil) | | | | |
| Inv 11 | Organic (tall | 99% | 60 | 0.27 | 73 |
| (Invention) | oil) | | | | |
| Inv 12 | Organic | 99.1% | 70 | 0.27 | 55 |
| (Invention) | (vegetable oil) | | | | |
| Inv 13 | Organic | 99.1% | 100 | 0.27 | 10 |
| (Invention) | (vegetable oil) | | | | |
| Inv 14 | Organic (tall | 99.1% | 100 | 0.27 | 13 |
| (Invention) | oil) | | | | |

From Table 3 it can easily be seen that when the acrylonitrile is of renewable origin then the amount of enzyme required is reduced.

By comparing counterexample CEx 4 and example Inv 10 (same quantity of enzyme), the quantity of residual acrylonitrile is reduced by a factor of more than 30.

By comparing counter-example CEx 4 and example Inv 9, one can see that approximately 25% less catalyst is needed to obtain the same residual amount of acrylonitrile at the end of the bioconversion.

### Example 3: Recycling of the Enzymatic Catalyst

The acrylonitrile bioconversion protocol described above is implemented with the difference that the enzyme introduced into the reaction medium comes from the filtration of the enzyme in suspension in the acrylamide solution obtained in Example 2.

In this example, the acrylonitrile has a renewable origin (tall oil) and contains a carbon-14 level of 80%.

The amount of residual acrylonitrile in the acrylamide solution resulting from the bioconversion is 97 ppm.

It is therefore possible to recycle the enzyme in the case of an acrylonitrile of renewable origin.

In contrast, the acrylonitrile bioconversion protocol of example Inv 8 is implemented with the difference that the enzyme introduced into the reaction medium is derived from the filtration of the enzyme suspended in the acrylamide solution obtained in counterexample CEx 2.

In this example, the acrylonitrile has a fossil origin. No acrylamide solution could be formed, the filtered enzyme is considered inactive.

Therefore, it is not possible to recycle the enzyme in the case of fossil-based acrylonitrile.

### Example 4: Synthesis of Ammonium Acrylate

A set of tests is carried out, adjusting the origin of the acrylamide, its percentage of ¹⁴C, as well as the dose of enzyme used to carry out examples Inv 16 to Inv 22 as summarised in Table 4.

The wt % of ¹⁴C is indicative of the nature of the carbon. The levels of ¹⁴C in the different acrylamides are measured according to the ASTM D6866-21 standard, method B. This standard makes it possible to characterize the bio-sourced nature of a chemical compound by determining the bio-sourced carbon level of said compound. A “zero pMC” represents the total absence of measurable ¹⁴C in a material, thus indicating a fossil carbon source.

### Protocol

In a 1,000 mL reactor equipped with a jacket, a stirrer and a condenser, 493 g of deionized water is added. The initial pH is adjusted to 7.5 with 10% sodium hydroxide.

The contents of the reactor are cooled to a temperature of 20° C. using a cryothermostat supplying the jacket of the reactor.

An amidase enzyme expressed by a Rhodococcus rhodochrous microorganism is added to the reaction medium. The enzyme has a dry extract of 10 wt %.

478 g of the acrylamide solution from the preceding examples is added continuously to the reactor at a rate of 61.7 g per hour.

The enzymatic conversion reaction of acrylamide is exothermic, the reactor is cooled by the jacket using the cryothermostat, so as to maintain a temperature of between 20 and 25° C. in the reaction medium.

At the end of the addition of the acrylamide solution, a ripening time of 1 hour is applied in order to convert a maximum of acrylamide. A sample of the reaction medium is taken for liquid chromatographic analysis to determine the amount of residual acrylamide.

The residual quantity of acrylamide must be less than 1000 ppm to validate the bioconversion test of acrylamide to ammonium acrylate.

TABLE 4

| | Origin of | wt % | Amount of | Residual amount of |
|---|---|---|---|---|
| | acrylamide | ¹⁴C | enzyme (g) | acrylamide (ppm) |
| CEx 5 | Counterexample | 0 | 8 | 24722 |
| | CEx 1 | | | |
| CEx 6 | Counterexample | 0 | 10 | 982 |
| | CEx 2 | | | |
| Inv 16 | Inv 1 | 80 | 8 | 632 |
| (Invention) | | | | |
| Inv 17 | Inv 2 | 80 | 6 | 948 |
| (Invention) | | | | |
| Inv 18 | Inv 3 | 80 | 10 | 45 |
| (Invention) | | | | |
| Inv 19 | Inv 4 | 60 | 8 | 764 |
| (Invention) | | | | |
| Inv 20 | Inv 5 | 70 | 8 | 702 |
| (Invention) | | | | |
| Inv 21 | Inv 6 | 100 | 8 | 110 |
| (Invention) | | | | |
| Inv 22 | Inv 7 | 100 | 8 | 80 |
| (Invention) | | | | |

The applicant observes that when the acrylamide is derived from acrylonitrile of renewable origin then the quantity of enzyme necessary is reduced.

By comparing counterexample CEx 6 and example Inv 18 (same amount of enzyme), the residual amount of acrylonitrile is reduced by a factor of more than 20.

By comparing counterexample CEx 6 and example Inv 17, one can see that 40% less catalyst is needed to obtain the same residual amount of acrylonitrile at the end of the bioconversion.

### Example 5: Bioconversion of Acrylonitrile to Ammonium Acrylate

A set of tests is carried out, adjusting the origin of the acrylonitrile, its percentage of ¹⁴C, as well as the dose of enzyme used to carry out examples Inv 23 to Inv 29 as summarised in Table 5.

The wt % of ¹⁴C is indicative of the nature of the carbon. The levels of ¹⁴C in the different acrylonitrile are measured according to the ASTM D6866-21 standard, method B. This standard makes it possible to characterize the bio-sourced nature of a chemical compound by determining the bio-sourced carbon level of said compound. A “zero pMC” represents the total absence of measurable ¹⁴C in a material, thus indicating a fossil carbon source.

The acrylonitrile of biological origin can come from the treatment of residues from the paper pulp industry (“tall oil” in English) in order to form the bio-propylene precursor before the ammoxidation process.

Alternatively, it may come from the processing of vegetable oil according to patent WO 2014/111598 or recycled cooking oil. The carbon 14 level in the different acrylonitriles is measured according to ASTM D6866-21 method B.

### Protocol

In a 1000 mL reactor equipped with a jacket, a stirrer and a condenser are added 621.5 g of deionized water. The initial pH is adjusted to 7.5 with 10% sodium hydroxide.

The contents of the reactor are cooled to a temperature of 20° C. using a cryothermostat supplying the jacket of the reactor.

A nitrilase enzyme expressed by a microorganism Rhodococcus rhodochrous is added to the reaction medium. The enzyme has a dry extract of 10 wt %.

373 g of acrylonitrile is continuously added to the reactor at a rate of 46.6 g per hour.

The enzymatic conversion reaction of acrylonitrile is exothermic, the reactor is cooled by the jacket using the cryothermostat, so as to maintain a temperature of between 20 and 25° C. in the reaction medium.

At the end of the addition of acrylonitrile, a ripening time of 1 hour is applied in order to convert a maximum of acrylonitrile. A sample of the reaction medium is taken for gas chromatographic analysis to determine the amount of residual acrylonitrile.

The residual quantity of acrylonitrile must be less than 1000 ppm to validate the bioconversion test of acrylonitrile to ammonium acrylate.

TABLE 5

| | | | Amount | Residual |
|---|---|---|---|---|
| | | % | of | amount of |
| | Acrylonitrile | weight | enzyme | acrylonitrile |
| | origin | ¹⁴C | (g) | (ppm) |
| CEx 7 | Fossil | 0 | 8 | 16425 |
| CEx 8 | Fossil | 0 | 10 | 992 |
| Inv 23 | Organic | 80 | 8 | 667 |
| (Invention) | (tall oil) | | | |
| Inv 24 | Organic | 80 | 6 | 923 |
| (Invention) | (tall oil) | | | |
| Inv 25 | Organic | 80 | 10 | 89 |
| (Invention) | (tall oil) | | | |
| Inv 26 | Organic | 60 | 8 | 666 |
| (Invention) | (tall oil) | | | |
| Inv 27 | Organic | 70 | 8 | 715 |
| (Invention) | (vegetable oil) | | | |
| Inv 28 | Organic | 100 | 8 | 113 |
| (Invention) | (vegetable oil) | | | |
| Inv 29 | Organic | 100 | 8 | 69 |
| (Invention) | (tall oil) | | | |

From Table 5, the applicant notes that when the acrylonitrile is renewable, then the amount of enzyme required is reduced.

By comparing counterexample CEx 8 and example Inv 25 (same amount of enzyme), the residual amount of acrylonitrile is reduced by a factor of more than 10.

By comparing counterexample CEx 8 and example Inv 24, one can see that 40% less catalyst is needed to obtain the same residual amount of acrylonitrile at the end of the bioconversion.

### Example 6: Preparation of a Solution of Bio-Acrylic Acid

In a 1000 mL reactor equipped with a jacket, a stirrer and a condenser are added 800 g of ammonium acrylate obtained in Example 22.

A 30% concentrated hydrochloric acid solution in water is added until a pH of 3 is obtained in the reaction medium.

The neutralization reaction is exothermic, the reactor is cooled by the jacket using the cryothermostat, so as to maintain a temperature of 20° C. in the reaction medium.

A solution of acrylic acid is thus obtained

### Example 7: Test of Biodegradability of Acrylamide Polymers P1 to P4

In a 2000 mL beaker, deionized water, monomers (see Table 6), 50 wt % sodium hydroxide solution (in water) are added. The solution thus obtained is cooled to between 5 and 10° C. and transferred to an adiabatic polymerization reactor. Nitrogen bubbling is carried out for 30 minutes in order to eliminate all traces of dissolved oxygen.

Are then added to the reactor:
0.45 g of 2,2′-azobisisobutyronitrile,
1.5 mL of an aqueous solution at 2.5 g/L of 2,2′-Azobis[2-(2-imidazolin-2-yl)propane] dihydrochloride,
1.5 mL of a 1 g/L aqueous solution of sodium hypophosphite,
1.5 mL of a 1 g/L aqueous solution of tert-butyl hydroperoxide,
1.5 mL of an aqueous solution at 1 g/L of ammonium sulphate and iron(II) hexahydrate (Mohr's salt).

After a few minutes the bubbling of nitrogen is stopped. The polymerization reaction then proceeds for 4 hours to reach a temperature peak. At the end of this time, the polymer gel obtained is chopped and dried, then again crushed and sieved to obtain a polymer in powder form.

The biodegradability (after 28 days) of the polymers thus obtained is evaluated according to the OECD 302B standard.

TABLE 6

| Polymer | P1 | P2 | P3 | P4 | CEx 8 | CEx 9 |
|---|---|---|---|---|---|---|
| Acrylamide | 276 | 276 | 276 | 276 | 276 | 276 |
| mass (g) | | | | | | |
| Monomer from | Inv 6 | Inv 8 | Inv 13 | Inv 15 | CEx 1 | CEx 4 |
| example | | | | | | |
| wt % ¹⁴C of | 100 | 80 | 70 | 100 | 0 | 0 |
| acrylamide | | | | | | |
| Mass of quaternised | 202.5 | 202.5 | 202.5 | 202.5 | 202.5 | 202.5 |
| dimethylaminoethyl | | | | | | |
| acrylate (g) | | | | | | |
| Mass of water (g) | 522 | $22 | 522 | 522 | 522 | 522 |
| % biodegradability | 51 | 42 | 50 | 40 | 12 | 15 |

The Applicant observes that the polymers obtained with bio-sourced monomers (containing ¹⁴C) are more easily biodegradable than the polymers having monomers of fossil origin.

### Example 8: Use of the Polymer as an Additive in a Papermaking Process

Retention agents are polymers added to cellulose fibre pulps prior to paper formation to increase paper retention efficiency.

Type of pulp used: Virgin fibre pulp:

A wet pulp is obtained by disintegrating a dry pulp to obtain a final aqueous concentration of 1 wt %. It is a neutral pH pulp consisting of 90% bleached virgin long fibres, 10% bleached virgin short fibres and 30% additional GCC (natural calcium carbonate) (Hydrocal® 55 from Omya) by weight on basis of fibre weight.

### Assessment of Total Retention and Load Retention

For all the following tests, the polymer solutions are prepared at 0.5 wt %. After 45 minutes of preparation, the polymer solutions are diluted 10 times before injection.

The different results are obtained using a Britt Jar type device with a stirring speed of 1000 rpm.

The process sequence is as follows:
T=0 s: Stirring of 500 mL of paper pulp at a concentration of 0.5 wt %.
T=10 s: Addition of the retention agent (300 g of dry polymer/tonne of dry pulp).
T=20 s: Elimination of the first 20 mL corresponding to the dead volume under the canvas, then recovery of 100 mL of white water.

The first pass retention percentage (% FPR), corresponding to the total retention, is calculated according to the following formula: % FPR=(C_{HB}−C_{WW})/C_{HB}*100

Percent first pass ash retention (% FPAR) is calculated using the following formula: % FPAR=(A_{HB}−A_{WW})/A_{HB}*100 with:
C_{HB}: Consistency of the headbox
C_{WW}: Consistency of white water
A_{HB}: Headbox ash consistency

For each of these analyses, the highest values correspond to the best performance.

### Evaluation of Gravity Drainage Performance Using the “Canadian Standard Freeness (CSF)”

In a beaker, the pulp is processed at a stirring speed of 1000 rpm.

The process sequence is as follows:
T=0 s: Stirring of 500 mL of paper pulp at a concentration of 0.6 wt %.
T=10 s: Addition of retention agent (300 g dry polymer/ton of dry pulp).
T=20 s: Stopping the stirring and adding the necessary quantity of water to obtain 1 litre.

This litre of dough is transferred to the “Canadian Standard Freeness Tester” and the TAPPI T227om-99 procedure is applied.

The volume, expressed in mL, and gives a measurement of gravity drainage. The higher the value, the better the gravity drainage.

This performance can also be expressed by calculating the percent improvement relative to the blank (% CSF). Higher values correspond to better performance.

The same polymers as before are tested and the results are presented below in Table 7.

TABLE 7

| Polymer | % FPAR | % FPR | % CSF |
|---|---|---|---|
| P1 | 33.5 | 74.3 | 15.3 |
| P2 | 30.1 | 69.9 | 10.1 |
| P3 | 33.4 | 74.1 | 15.2 |
| P4 | 26.5 | 68.8 | 9.6 |
| CEx 8 | 20.3 | 64.2 | 1.5 |
| CEx 9 | 20.7 | 64.8 | 2 |

The applicant observes that the polymers obtained with bio-sourced monomers (containing ¹⁴C) have better performance in terms of drainage and retention than the polymers having monomers of fossil origin.

### Example 9: Measurement of the Degree of Insolubility in Polymer Solutions

UL viscosity (Brookfield viscosity), insolubility rate and insolubility point are measured on a polymer composed of 70 mole % acrylamide and 30 mole % quaternised DMAEA, prepared by conventional bulk polymerization.

UL viscosity is measured using a Brookfield viscometer fitted with a UL adapter, the unit of which rotates at 60 rpm (0.1 wt % of polymer in a saline solution of 1M sodium chloride) between 23 and 25° C.

The insolubility rate is measured by transferring 1 g of the polymer solution into 200 mL of water at 20° C., stirring for 2 hours, then the dissolved solution is filtered with a 4 cm diameter filter with a porosity of 200 μm. After complete draining of the filtered solution, the filter paper is weighted. In the case of a non-filterable solution, the screen filter is placed at 105° C. for 4 hours. The residual mass is used to determine the insoluble quantity, the insolubility rate is related to the initial mass of the polymer. The vinyl acrylate impurity creates covalent bonds between 2-dimethylaminoethyl acrylate monomers, resulting in aggregates that do not pass through the filter.

The insolubility point corresponds to the number and size of the aggregates on the filter. The following scale is used: point (pt) between 1 and 3 mm; big dot (bp) for more than 3 mm (visual count).

The polymers that have been prepared previously are tested and the results are summarized in Table 8.

TABLE 8

| | Viscosity | Number of insoluble | Insoluble |
|---|---|---|---|
| Polymer | UL (Cps) | (points) | content (%) |
| P1 | 5.3 | 5 | 0 |
| P2 | 5.3 | 8 | 2 |
| P3 | 5.4 | 6 | 1 |
| P4 | 5.3 | 10 | 3 |
| CEx 8 | 5.1 | 30 | 7 |
| CEx 9 | 5.2 | 15 | 7 |

Polymers that are obtained with bio-sourced monomers (containing carbon 14) have better solubility than polymers with monomers of fossil origin.

### Example 10: Measurement of Friction Reduction

The polymers P1 to P4 and CEx8 to CEx9 are dissolved with stirring at a concentration of 10,000 ppm in a brine composed of water, 85 g of sodium chloride (NaCl) and 33.1 g of calcium chloride (CaCl₂), 2H₂O) per litre of brine. The polymer saline solutions thus obtained are then injected at a concentration of 0.5 pptg (parts per billion/gallon) into the brine circulated for the Flow Loop tests.

Indeed, to evaluate the friction reduction of each of the polymers P1 to P4 and CEx8 to CEx9, the reservoir of the loop of the Flow Loop (calibrated tube length (loop): 6 m, internal diameter of the tube: 4 mm) is filled with 20 L of brine (as described above). The brine is then circulated through the Flow Loop at a rate of 24 gallons per minute. The polymer is added at a concentration of 0.5 pptg in the same recirculating brine. The percentage of friction reduction is thus determined thanks to the measurement of pressure variations measured inside the Flow Loop.

The graphs in FIGS. 2 and 3 represent the percentage reduction in friction as a function of time for the brine containing each of the polymers.

Friction reduction is improved when the brine contains polymers P1 to P4 (compared to polymers CEx8 to CEx9).

### Example 11: Evaluation of the Biodegradability of Polymers of Acrylic Acid

In a 2000 mL beaker, deionized water, monomers (see Table 9), 50 wt % sodium hydroxide solution (in water) are added.

The solution thus obtained is cooled to between 5 and 10º C. and transferred to an adiabatic polymerization reactor. Nitrogen bubbling is carried out for 30 minutes in order to eliminate all traces of dissolved oxygen.

Are then added to the reactor:
0.45 g of 2,2′-azobisisobutyronitrile,
1.5 mL of an aqueous solution at 2.5 g/L of 2,2′-Azobis[2-(2-imidazolin-2-yl)propane] dihydrochloride,
1.5 mL of a 1 g/L aqueous solution of sodium hypophosphite,
1.5 mL of a 1 g/L aqueous solution of tert-butyl hydroperoxide,
1.5 mL of an aqueous solution at 1 g/L of ammonium sulphate and iron(II) hexahydrate (Mohr's salt).

After a few minutes the bubbling of nitrogen is stopped. The polymerization reaction then proceeds for 4 hours to reach a temperature peak. At the end of this time, the polymer gel obtained is chopped and dried, then again crushed and sieved to obtain a polymer in powder form.

The biodegradability (after 28 days) of the polymers thus obtained is evaluated according to the OECD 302B standard.

TABLE 9

| Polymer | P5 | P6 | P7 | P8 | CEx10 | CEx11 |
|---|---|---|---|---|---|---|
| Mass acrylic acid (g) | 30 | 66 | 102 | 30 | 30 | 102 |
| Monomer from example | 21 | 22 | 28 | 19 | CEx 6 | CEx 8 |
| wt % ¹⁴C of acrylic acid | 100 | 100 | 100 | 100 | 0 | 0 |
| oligomer | | | | | | |
| Mass of acrylamide (g) | 330 | 384 | 396 | 330 | 330 | 396 |
| wt % carbon 14 of | 0 | 0 | 0 | 0 | 0 | 0 |
| acrylamide | | | | | | |
| Mass of 2-acrylamido-2- | 105 | 42 | 0 | 105 | 105 | 0 |
| methylpropane sulfonic | | | | | | |
| acid (g) | | | | | | |
| wt % ¹⁴C of 2- | 0 | 0 | 0 | 0 | 0 | 0 |
| acrylamido-2- | | | | | | |
| methylpropane | | | | | | |
| sulfonic acid | | | | | | |
| Mass of washing soda at | 42 | 19 | 4 | 42 | 45 | 11 |
| 50% (g) | | | | | | |
| Mass of water (g) | 493 | 489 | 498 | 493 | 490 | 491 |
| % biodegradability | 15 | 30 | 45 | 21 | 5 | 10 |

The P5 to P8 polymers, which are obtained with bio-sourced monomers (containing C¹⁴) are more easily biodegradable than the counterexamples of fossil origin.

### Example 12: Measurement of Filtration Coefficients

Filtration tests are carried out on polymers P5 to P8 and CEx10 to CEx11. The polymers are put into solution at a concentration of 1000 ppm in a brine containing water, 30,000 ppm of NaCl and 3,000 ppm of CaCl₂)·2H₂O. Filtration quotients (FR) are measured on filters having a pore size of 1.2 μm representative of low permeability oil deposits. The results are shown in Table 10.

TABLE 10

| Polymer | Filtration Quotient |
|---|---|
| P5 | 1.09 |
| P6 | 1.08 |
| P7 | 1.07 |
| P8 | 1.08 |
| CEx 10 | 1.23 |
| CEx 11 | 1.3 |

The filtration quotients (FR) are lower for the P5 to P8 polymers (compared to the CEx 10 to CEx 11 polymers).

### Example 13: Test of Resistance to Chemical Degradation

Tests of resistance to chemical degradation of polymers P5 to P8 and CEx10 to CEx11 were carried out under aerobic conditions in the presence of different concentrations of iron (II) (2, 5, 10 and 20 ppm) in a brine composed of water, 37,000 ppm NaCl, 5,000 ppm Na₂SO₄ and 200 ppm NaHCO₃. The polymers are dissolved at a concentration of 1000 ppm in brines containing Iron (II). The results of the degradation tests (table 11) are obtained after 24 hours. Each percentage loss of viscosity is determined by comparing the viscosity of the polymer solution in the brine after dissolution of the polymer (to) and its viscosity after 24 h (t_{24 h}). The viscosities are measured with a Brookfield viscometer (UL module, 25° C., 60 rpm-1).

TABLE 11

| | Iron (II) concentration | | | |
|---|---|---|---|---|
| Polymer | 2 ppm | 5 ppm | 10 ppm | 20 ppm |
| | % loss of viscosity | | | |
| P5 | 3 | 7 | 10 | 13 |
| P6 | 5 | 8 | 12 | 15 |
| P7 | 3 | 6 | 9 | 12 |
| P8 | 2 | 5 | 8 | 10 |
| CEx 10 | 10 | 15 | 21 | 32 |
| CEx 11 | 14 | 18 | 25 | 35 |

Polymers P5 to P8 are more resistant to chemical degradation than polymers CEx10 to CEx11.

1. A biological method for obtaining an MO monomer comprising an ethylenic unsaturation by bioconversion of a CN compound comprising at least one nitrile function, said CN compound being at least partially renewable and non-fossil, said biological method comprising at least one step of enzymatic bioconversion of the CN compound in the presence of a biocatalyst comprising at least one enzyme.

2. The method according to claim 1, wherein the CN compound has a bio-sourced carbon content of between 5 wt % and 100 wt % relative to the total carbon weight in the CN compound, the bio-sourced carbon content being measured according to a standard ASTM D6866-21 Method B.

3. The method according to claim 1, wherein the CN compound is (meth)acrylonitrile or 3-hydroxypropionitrile.

4. The method according to claim 1, wherein the MO monomer has a bio-sourced carbon content of between 5 wt % and 100 wt % relative to the total carbon weight in said MO monomer, the bio-sourced carbon content being measured according to a standard ASTM D6866-21 Method B.

5. The method according to claim 1, wherein the MO monomer is selected from the group consisting of (meth)acrylamide, ammonium (meth)acrylate, and (meth)acrylic acid.

6. The method according to claim 1, wherein the CN compound and/or the MO monomer are fully renewable and non-fossil.

7. The method according to claim 1, wherein the MO monomer is (meth)acrylamide, the CN compound is (meth)acrylonitrile, and in that the biocatalyst comprises at least a nitrile hydratase enzyme or at least a nitrilase enzyme.

8. (canceled)

9. The method according to claim 1, wherein the MO monomer is a (meth)acrylate salt, the CN compound is (meth)acrylamide, and in that the biocatalyst comprises at least one amidase enzyme, said CN (meth)acrylamide monomer having been previously obtained by bioconversion of (meth)acrylonitrile that is at least partially renewable and non-fossil according to a biological method comprising at least one step of enzymatic hydrolysis of said (meth)acrylonitrile in the presence of a biocatalyst comprising at least one nitrile hydratase enzyme.

10. The method according to claim 8, further comprising: wherein converting acrylate or methacrylate salt respectively into acrylic acid or methacrylic acid.

11. The method according to claim 1, wherein the CN compound is derived from a recycling process.

12. (canceled)

13. (canceled)

14. A bio-(meth)acrylamide obtained by bioconversion of (meth)acrylonitrile that is at least partially renewable and non-fossil, said bioconversion comprising at least one step of enzymatic hydrolysis of said (meth)acrylonitrile in the presence of a biocatalyst comprising at least one nitrile hydratase enzyme.

15. (canceled)

16. A bio-(meth)acrylate salt obtained by bioconversion of (meth)acrylonitrile that is at least partially renewable and non-fossil, said bioconversion comprising at least one step of enzymatic hydrolysis of said (meth)acrylonitrile in the presence of a biocatalyst comprising at least one nitrilase enzyme.

17. (canceled)

18. (canceled)

19. A polymer obtained by polymerization of at least one MO monomer obtained by the method according to claim 1.

20. The polymer according to claim 19, wherein the polymer is a copolymer of:
at least a first MO monomer obtained by the method according to claim 1,
at least a second monomer different from the first monomer, said second monomer is selected from the group consisting of nonionic monomers, anionic monomers, cationic monomers, zwitterionic monomers, monomers comprising a hydrophobic moiety, and mixtures thereof.

21. The polymer according to claim 19, wherein the polymer is a copolymer comprising:
at least 5 mol %, preferably at least 10 mol %, preferentially between 20 mol % and 90 mol %, more preferentially between 30 mol % and 99 mol % of a first monomer, said monomer being an MO monomer obtained by the method according to claim 1, and
at least 1 mol %, preferentially between 5 mol % and 90 mol %, more preferentially between 10 mol % and 80 mol % of at least one second monomer comprising an ethylenic unsaturation, said second monomer being different from the first monomer, and comprising a bio-sourced carbon content of between 5 wt % and 100 wt %, preferably from 10 wt % to 100 wt %, relative to the total carbon weight in said second monomer, the bio-sourced carbon content being measured according to a standard ASTM D6866-21 Method B.

22. The polymer according to claim 21, wherein said at least second monomer is selected from an oligomer of acrylic acid, 2-acrylamido-2-methylpropane sulfonic acid (ATBS) and/or a salt thereof, N-vinylformamide (NVF), N-vinylpyrrolidone (NVP), dimethyldiallylammonium chloride (DADMAC) quaternized dimethylaminocthyl acrylate (ADAME), quaternized dimethylaminoethyl methacrylate (MADAME), a substituted acrylamide having the formula CH₂═CHCO—NR¹R², R¹ and R² being, independently of each other, a linear or branched carbon chain CₙH₂ₙ₊₁, wherein n is between 1 and 10.

23. (canceled)

24. (canceled)

25. (canceled)

26. A method for enhanced oil and/or gas recovery by sweeping a subterranean formation, comprising:
a. preparing an injection fluid from a polymer, according to claim 19, with water or brine,
b. injecting the injection fluid into a subterranean formation,
c. sweeping the subterranean formation with the injected fluid,
d. recovering an aqueous mixture of oil and/or gas.

27. A method for hydraulic fracturing of subterranean oil and/or gas reservoirs, comprising:
a. preparing an injection fluid from a polymer, according to claim 19, with water or brine, and with at least one proppant,
b. injecting said fluid into the subterranean reservoir and fracturing at least a portion thereof to recover oil and/or gas.

28. A method for drilling and/or cementing a well in a subterranean formation, comprising:
a. preparing a fluid from a polymer according to claim 19, with water or brine,
b. injecting said drilling and/or cementing fluid into the subterranean formation via the drill head in at least one step of drilling or cementing a well.

29. A method for making a sheet of paper, cardboard or the like, whereby, before forming said sheet, at least one polymer according to claim 19 is added to a fiber suspension at one or more injection points.

30. (canceled)

31. (canceled)

32. (canceled)

33. (canceled)
